(19) United States
(12) Patent Application Publication
(10) Pub. No.: US 2026/0021285 A1
(43) Pub. Date: Jan. 22, 2026

(54) FOLDABLE ANTI-MICROBIAL TUBING PROTECTION SYSTEM, AND DEVICES AND METHODS THEREOF

(71) Applicant: I+D Device Solutions LLC, Corona, CA (US)

(72) Inventors: Iris GERETSCHNIG, Corona, CA (US); Gregory TUDRYN, San Diego, CA (US); Desiree VALDEZ, Loma Linda, CA (US)

(21) Appl. No.: 19/288,876

(22) Filed: Aug. 1, 2025

Related U.S. Application Data
(63) Continuation of application No. PCT/US2024/014313, filed on Feb. 2, 2024.
(60) Provisional application No. 63/483,254, filed on Feb. 3, 2023.

(51) Int. Cl. A61M 39/16; A61M 25/02

(52) U.S. Cl. CPC A61M 39/165; A61M 25/02; A61M 2205/584

(57) ABSTRACT

Embodiments described herein relate to encasements for protection of central lines, arterial lines, and/or intravenous (IV) lines from contaminants, and methods of making and using the same. In some embodiments, an encasement device can include a flexible sleeve that fits over a central line and protects the central line from contaminants. In some embodiments, the flexible sleeve can include one or more foldable sections.

### CROSS REFERENCE TO RELATED APPLICATIONS

This application is a continuation of International Application No. PCT/US2024/014313, filed Feb. 2, 2024, and titled “Foldable Anti-Microbial Tubing Protection System, and Devices and Methods Thereof,” which claims priority to and the benefit of U.S. Provisional Patent Application No. 63/483,254, filed Feb. 3, 2023, and titled “Foldable Anti-Microbial Tubing Protection System, and Devices and Methods Thereof,” the disclosures of both of which are hereby incorporated by reference in their entireties.

### TECHNICAL FIELD

The present disclosure relates generally to tubing encasements or coverings, and methods of making and using the same, and more specifically, to protective coverings for intravenous (IV) tubing, catheter tubing, IV ports, IV connector sites or other types of tubing used in a medical setting.

### BACKGROUND

Central line-associated bloodstream infections (CLABSI) are one of the most expensive healthcare associated infections (HAI) despite being highly preventable. CLABSI reduction can lower preventable morbidity and mortality as well as significantly decrease unnecessary healthcare costs. The Centers for Disease Control and Prevention (CDC) has made reducing HAIs a top priority, yet despite healthcare workers' adherence to CDC guidelines for CLABSI prevention, CLABSI rates continue to increase and have been exacerbated by the COVID-19 pandemic. CLABSI's are commonly caused by microbes such as bacteria, fungi, and viruses including, but not limited to Staphylococci, Enterococci, Staph aureus, Klebsiella, Enterobacter, Pseudomonas, E. coli, and Candida on medical equipment and/or lines, as well as patient's skin and surrounding surfaces. These microbes can migrate into the bloodstream via catheter sites and cause infections. Solutions are therefore needed that reduce the migration of such microbes near catheter sites.

### SUMMARY

Embodiments described herein relate generally to encasements for protecting of central lines, IV lines, IV ports, and/or connection sites from contaminants, and methods of making and using the same. In some embodiments, an encasement device can include a flexible sleeve that fits over IV tubing, IV ports, and connection sites and protects from contaminants. The flexible sleeve has a first open end and a second open end. First and second closing elements at the open ends can be configured to close the open ends. In some embodiments, the first closing element can secure the first open end to a medical dressing. In some embodiments, the tubing can include a central line, an IV line, a peripherally inserted central catheter (PICC) line and/or a hemodialysis line. In some embodiments, the encasement device can include a movable flap coupled to the flexible sleeve. The flap can open to allow access to the tubing and any other components connected to the tubing and the flap can close to protect the tubing and such components from contaminants.

In some aspects, a device includes a flexible sleeve sized to receive and enclose a set of one or more medical lines connected to a patient, the flexible sleeve having first and second ends and a flexible body extending between the first and second ends. The flexible sleeve includes one or more antimicrobial agents disposed throughout an entire length of the flexible body. In some embodiments, the antimicrobial agent can be embedded into the flexible sleeve. In some embodiments, the antimicrobial agent can be coated onto the flexible sleeve. The device includes a first closing element disposed at the first end of the flexible sleeve. The first closing element selectively opens and closes the first end of the flexible sleeve. The second closing element is disposed at the second end of the flexible sleeve. The second closing element selectively opens and closes the second end of the flexible sleeve. The first and second closing elements open the first and second ends of the flexible sleeve such that the flexible sleeve can be placed around the set of medical lines. The first and second closing elements close the first and second ends of the flexible sleeve around the set of medical lines at the first and second ends of the flexible sleeve such that the flexible sleeve is configured to protect the set of medical lines from contaminants.

In some aspects, a device includes a flexible sleeve sized to receive and enclose a set of one or more medical lines connected to a patient. The flexible sleeve has first and second ends and a flexible body extending between the first and second ends. The device includes a first closing element disposed at the first end of the flexible sleeve. The first closing element selectively opens and closes the first end of the flexible sleeve. The device includes a second closing element disposed at the second end of the flexible sleeve. The second element selectively opens and closes the second end of the flexible sleeve. The first and second closing elements open the first and second ends of the flexible sleeve such that the flexible sleeve can be placed around the set of medical lines. The first and second closing elements close the first and second ends of the flexible sleeve around the set of medical lines at the first and second ends of the flexible sleeve such that the flexible sleeve is configured to protect the set of medical lines from contaminants. The device further includes a perforation line formed in the flexible sleeve and extending along a longitudinal length of the flexible sleeve. The perforation line allows the flexible sleeve to be opened along the longitudinal length of the flexible sleeve such that the flexible sleeve can be removed from the set of medical lines.

In some aspects, a device includes a flexible sleeve sized to receive and enclose a set of medical lines connected to a patient. The flexible sleeve has first and second ends and a flexible body extending between the first and second ends. The set of medical lines includes a catheter terminating in one or more connectors and one or more lines configured to couple to the one or more connectors. The device includes at least one closing element, the at least one closing element disposed at the first or second end of the flexible sleeve and configured to selectively open and close the first or second end. The at least one closing element opens the first or second end of the flexible sleeve such that the flexible sleeve can be placed around the set of medical lines. The at least one closing element closes the first or second end of the flexible sleeve around the set of medical lines at the first or second end of the flexible sleeve such that the flexible sleeve is configured to protect the set of medical lines from contaminants. The device includes a lateral opening closing element configured to reversibly close a lateral opening formed in the flexible body at a location near the one or more connectors. The lateral opening closing element opens to allow access to the one or more connectors. The lateral opening closing element closes to protect the set of medical lines from contaminants.

In some aspects, a device includes a flexible sleeve sized to receive and enclose a set of one or more medical lines connected to a patient. The set of medical lines includes a medical line disposed through an insertion site. The flexible sleeve has first and second ends and a flexible body extending between the first and second ends, the first end of the flexible sleeve defining an opening and the second end of the flexible sleeve with a closing element or being permanently closed. The device includes a flange disposed at the first end of the flexible sleeve, the flange surrounding the opening and having an adhesive surface. The adhesive surface directly attaches to a dressing or surface at the insertion site such that the flange encircles the catheter and the flexible sleeve closes around the set of medical lines. The adhesive surface detaches from the dressing or surface in response to a peeling force applied by a user to the flange.

In some embodiments, a device includes a flexible sleeve with one or more foldable parts that is configured to enclose a set of one or more medical lines connected to a patient. In some embodiments, the flexible sleeve can include two parts, e.g., a top part that can fold over a bottom part. In some embodiments, the flexible sleeve can include three parts, e.g., a bottom layer, a top layer that can fold over the bottom layer, and a window. Each layer or window may include a tab for manipulating the layers independently.

### BRIEF DESCRIPTION OF THE DRAWINGS

FIG. 1 is a block diagram of an encasement for protection of one or more lines, ports, and/or connection sites (e.g., IV tubing) from contaminants, according to an embodiment.

FIG. 2 is an illustration of an encasement for protection of one or more lines from contaminants, according to an embodiment.

FIGS. 3A and 3B are illustrations of an encasement with a flap for protection of one or more lines from contaminants, according to an embodiment.

FIG. 4 is an illustration of an encasement for protection of one or more lines from contaminants, according to an embodiment.

FIG. 5 is an illustration of an encasement with a flap for protection of one or more lines from contaminants, according to an embodiment.

FIGS. 6A and 6B are illustrations of a collection of packs for packaging of encasements, according to an embodiment.

FIGS. 7A and 7B are illustrations of a system for dispensation of encasement packs, according to an embodiment.

FIGS. 8A-8G are illustrations of a method of protecting a line from contaminants, according to various embodiments.

FIG. 9 is a block diagram of a method of protecting a line from contaminants, according to an embodiment.

FIG. 10 is an illustration of a closing element of an encasement, according to an embodiment.

FIG. 11 is an illustration of a mounting loop of an encasement, according to an embodiment.

FIGS. 12A-C are illustrations of encasements for protection of one or more lines from contaminants, according to various embodiments.

FIGS. 13A and 13B are illustrations of an encasement with adhesive strips for

securement to surfaces, according to various embodiments.

FIG. 14 is an illustration of an encasement with perforations, according to an embodiment.

FIGS. 15A and 15B are illustrations of encasements with holders, according to various embodiments.

FIG. 16 is an illustration of an encasement in a collapsed and folded configuration, according to an embodiment.

FIGS. 17A-17E are illustrations of an encasement implemented as a foldable sleeve with two parts, according to an embodiment.

FIGS. 18A-18F are illustrations of an encasement implemented as a foldable sleeve with three parts, according to an embodiment.

FIGS. 19A-19D are illustrations of an encasement implemented as a foldable sleeve, according to an embodiment.

FIGS. 20A-20C are illustrations of an encasement implemented as a foldable sleeve, according to an embodiment.

FIG. 21 is an illustration of an encasement implemented as a foldable sleeve in an open configuration, according to an embodiment.

### DETAILED DESCRIPTION

Bloodstream infections in patients with central lines are a costly and preventable danger to patient health and safety. A central line is a tube placed into a large vein of a patient in order to administer medical treatment and can potentially be a major point of contamination or infection. HAI's cause significant loss of life and financial burden in the healthcare community. Some of the most dangerous HAI's are CLABSI's and catheter-related bloodstream infections (CRBSI). According to the CDC, up to one in four people who contract CLABSI's or CRBSI's die. Estimates show the average cost to treat CLABSI is more than $45,000 per infection. Preventing CLABSI's can include a focus on protecting the central line both during its insertion and its maintenance, both of which present opportunities for the introduction of microorganisms.

Many variables can contribute to bloodstream infections, including intrinsic risk factors related to the patient's age or underlying diseases, and extrinsic risk factors such as lack of maximal sterile barriers during insertion, exposure to environmental contaminates, and multiple central venous catheter ports. Handling of the central lines and ports after insertion increases the risk of infection. CLABSI reduction initiatives are often successful when they eliminate as many variables as possible. This can be a three-prong approach, that includes the people in contact with the patient, the standards of the healthcare facility, and the technology employed in the implementation of the central line. These three factors can each be important and are often seen as interdependent.

Effective products can aid medical professionals in reducing infections. Products that are easy to use and designed to help medical professionals comply with industry best practices and standards are important for crucial patient care tasks. Such tasks can include securing catheters and protecting IV lines from contamination. In some cases, a blend of products working in concert, alongside evolving procedures and standards provide a scenario to reduce infection rates.

Conventional medical barriers and dressings can be used in many contexts, including wound dressings, post-surgical dressings, and other specialized situations. Catheters allow the drainage of fluids, injection of fluids, or access by surgical instruments (e.g., arterial lines for angioplasty). Problems occur with prolonged use of a catheter/IV or drainage tube, as the prolonged use allows for biofilm formation and/or creates a site for contamination both at the surface and at access ports of the IV, which are often exposed to touch and airborne contaminations. Conventional barriers often protect a wound or catheter/IV site against microbes and infections. In some instances, the barrier can cover the catheter entry point, but surrounding areas (such as IV access points and proximal areas during IV use) are susceptible to contamination, such as IV access points and proximal areas during IV use. Catheter/IV manipulation, patient transport, and environmental factors including bodily fluids, surface contact and IV manipulation in between routine cleaning procedures often allow vectors for contamination and infection that are difficult to practically mitigate with intermittent cleaning procedures. Cleaning procedures often only include daily chlorhexidine skin cleaning alongside constant re-positioning of IV lines in an attempt to keep them clear of contaminated areas. There are no widely adopted protocols for cleaning IV lines. Ports are usually cleaned with alcohol or povidone-iodine (e.g., Betadine®) before and after use.

Short-term catheters and long-term catheters can be non-tunneled or tunneled. A short-term, non-tunneled catheter can stay in place for days or weeks. A long-term tunneled catheter can stay in place for months or years. Examples of both short-term and long-term catheters include Central Venous Catheters (CVC), PICC lines, hemodialysis catheters, apheresis catheters, and arterial line catheters. Catheters are inserted under the skin into a large vein or artery. The catheter lumens and ports exit outside of the body and allow for vascular access for administration of fluid, blood, medication, and/or nutrition, and/or for removal of fluid and/or blood. Arterial lines are catheters inserted into an artery either on the wrist of an arm or in the groin area to monitor the blood pressure, for withdrawing blood and/or to infuse a medication to break down a blood clot that occludes an artery.

Each entry into the access points in the delivery systems is an opportunity to introduce microorganisms. The post-CVC insertion period presents multiple opportunities for the risk of infection. The proper maintenance of lines is important for continued patient safety. Recent reports to the National Healthcare Safety Network (NHSN) by Pennsylvania's acute care hospitals assert that nearly 72% of al CLABSI's occurred more than 5 days after insertion. This suggests that infection prevention lapses oftentimes occur in the post insertion care and maintenance of the CVC's.

When not in use, catheter ports are cleaned and covered with a disinfecting cap, and the line is clamped. However, IV lines and IV connection sites are left exposed to microbes on skin and the surrounding environment. The hubs on central venous catheters (CVC) are a common source of bacterial colonization and serve as an immediate portal of entry for microorganisms to the intraluminal surface of the catheter. These colonizers from the catheter hub and lumen can be dispersed into the bloodstream resulting in CLABSI. In long term CVC's, the needleless connectors (NC) and catheter hubs are more frequently accessed and can potentially lead to CLABSI's. The disinfection of a catheter hub surface every time before it is accessed is therefore important.

Exposed IV lines are widely recognized as a point for contamination from routine handling, bodily fluids, vomit, and excrement, which can each harbor a variety of microorganisms that may cause infection, if handling the port area. While aseptic technique per CDC requires that the CLABSI prevention protocol is followed, in practicality, patients may have a line in a complex position, complex line routing (e.g., around a groin, armpit, etc.), and/or an area of repeated exposure to contaminants. The biofilm contamination may lead to CLABSI when microbes migrate to ports or when microbes are transferred onto the ports when contaminated IV lines are handled and the port is accessed.

Preparation(s) or cleaning(s) of the insertion site, the dressing and the IV lines often includes cleansing with alcohol, chlorhexidine, or similar chemicals. This can be a preparation routine for surgery or for daily routines. There is currently no protocol for cleaning the lines themselves. Presently, IV lines and IV connection sites are protected by wiping the skin once a day with chlorhexidine and by moving lines away from obvious sources of contamination. Access ports can be cleaned with alcohol, and disinfecting caps (e.g., Curos™ caps) can be included in a delivery system. Cleaning can be done when injecting or drawing though the port, and another disinfecting cap is added to the port. Skin cleaning is often only required or done once per day, allowing buildup of potential biofilm on the medical lines and surrounding areas. Relying on once daily skin decontamination protocols to protect IV lines is ineffective when IV lines are in an inherently contaminated environment (armpits/groins/etc.). Providing continuous IV protection against exposure to skin, body fluids, and many other contaminates offers reduction or prevention against biofilm formation and/or transfer. Most antimicrobial products are incorporated into dressings or seals primarily focusing on the skin-to-IV insertion site contact point. Presently, there are no IV covering solutions for IV's in use. Existing solutions do not incorporate continuous antimicrobial protection of the exposed IV line. Disinfecting caps and advanced dressings can aim to solve some issues but fail to address problems concerning clean lines and provide facile access to lines. Embodiments described herein can work with or without adjacent solutions to increase infection prevention.

Prolonged and continuous protection of IV lines while retaining intuitive functional access can be desirable. Preventing CLABSI in an efficacious and cost-effective manner without compromising physical access of IV lines in use is also important.

Embodiments described herein generally relate to IV and/or catheter encasements or coverings, systems and components thereof, and methods of making the same. In some embodiments, encasements described herein can include sleeves (e.g., coverings with open ends or multiple openings) or bags (e.g., coverings or containers with an opening at one end). In some embodiments, encasements described herein can include sleeves with open ends permitting IV-line entry and exit and closing elements (e.g., cinch elements) at both ends to facilitate selective closure and opening, (e.g., to permit line access). Closing elements can allow for access to the IV and/or catheter, removal of the encasement, and/or re-closing. In some embodiments, adhesive flanges can be included on none, one, or both ends of a sleeve. In some embodiments, a sleeve can include an adhesive flange at a first open end and a closure element at a second open end. The encasements described herein can be used to protect critical points and areas of the IV line that are prone to contamination. These can include entry points, port access points, dressing entry points, and/or other critical points and areas of the IV line.

In some embodiments, an encasement can be composed of a polymer or other suitable flexible material. In some embodiments, the encasement can be composed of a thermoplastic material capable of forming an effective barrier to contamination and being subjected to repeated manipulation and/or adjustments during the lifetime of the use of the sleeve. In some embodiments, the thermoplastic material can be embedded with, layered, and/or coated with one or more antimicrobial materials or moieties. In some embodiments, the antimicrobial materials can include bioactive materials, bioactive metals, silver, copper, zinc, nickel, cobalt, molybdenum, an oligodynamic metal, an organic antimicrobial, an organic moiety formed of one or more essential oils, an ionic polymer, an ionic oligomer, an ionic rubber, an organometallic material, effective antimicrobial organic compounds, or any other suitable antimicrobial materials or combinations thereof.

In some embodiments, the encasement can extend beyond critical points of access and contamination for additional protection and coverage of IV lines, ports, and/or sites of manipulation. In some embodiments, the encasement can include holders for mounting and/or securement of the IV lines in a desired position and/or configuration relative to the encasement. In some embodiments, the encasement can include adhesive strips on the encasement for securing on a desired surface. In some embodiments, a flap (e.g., an access point, window, and/or door) can be included on the sleeve. The flap can permit repeated access to IV lines, ports, and/or key areas between the terminal ends of the encasement without necessitating removal of the encasement. In some embodiments, the flap can be resealable, permitting re-closure of the encasement barrier and protection of IV lines during or after IV access or activity.

Methods described herein can address several drawbacks of existing solutions for reducing IV line infections. These can include, for example, coverage of IV in use, while allowing central lines to remain active and/or readily accessible. These can also include, for example, ease of access to IV points that are often critical areas of contamination if uncovered. Durability for continued use while IV lines are active, in use, or in between medical treatment can be an additional improvement. Antimicrobial activity towards continuous exposure in areas of high contamination risk (e.g., saliva, feces, vomitus, phlegm, food, skin, gown, linen, surface and floor contact, etc.) can be reduced using the methods described herein. For example, the closure of the ends of a sleeve with drawstrings, adhesives, and/or seals can permit repeated facile access to IV lines without requiring removal of the IV enclosure. In some embodiments, an adjustable adhesive flange can work in concert with dressings to form an effective contiguous barrier to infection.

Encasements described herein can be configured to provide antimicrobial or infection reduction properties during prolonged use while maintaining ease of IV line access during treatment. As a result of these advantageous properties, the encasements prepared according to the disclosed methods can be used with a variety of medically useful articles. In some embodiments, the disclosed encasements can be used to provide durable and more effective barriers to CLABSI, either alone or in combination with existing dressings. Additionally, the disclosed encasements can provide more effective access to lines when in use when compared to standard IV coverings, wraps, or dressings.

CDC guidelines for reducing infections focus on practices for inserting central lines, maintaining and handling lines, and removal of unnecessary lines. Devices described herein are configured to encase central line access ports and/or tubing in an antimicrobial environment to prevent prolonged exposure to pathogens found on a patient's skin and in surrounding environment. In some embodiments, a sterile sleeve can be configured to slide over central line connection sites, access ports, and IV tubing and to encase or cover such components to decrease biological burden on these potential pathogen entry points. In some embodiments, the outside of the sleeve can be antimicrobial and/or act as a physical barrier to prevent access ports and IV tubing from coming into direct contact with potential pathogens on the patient's skin and in the environment. In some embodiments, the sleeve can cover the central line connection sites closest to the patient, run the length of the central line, and extend to where the tubing connects to the IV pump. In some embodiments, both ends of the sleeve can be drawn closed, adhesively sealed, and/or cinched closed to keep them in place. In some embodiments, the sleeve can be formed of a flexible material such as, for example, a polymer.

Embodiments described herein can also be used on peripheral lines, which may not impose as high a risk for infection but can nevertheless have benefits from inclusion of an encasement. While several of the embodiments described herein are described in reference to hospital settings, it can be appreciated that such embodiments can be used for patients outside of the hospital, such as those receiving infusions (e.g., on a daily basis) at home. In such applications, the lines can be constantly covered or covered for a prolonged period of time. Where disposal of the encasement device is necessary, e.g., in environments outside of a hospital, embodiments described herein may utilize a biodegradable variant to minimize ecological impact.

Encasement devices such as pouches, bags, and/or sleeves described herein can also be suitable for children or pediatric patients with lines, e.g., in a hospital or at home. Children can be more active, and therefore risk of infection associated with children can be higher. Use of antimicrobial infused IV lines and catheters can be less effective in pediatric patients, whereas the physical barrier formed by the encasements described herein can provide higher efficacy in preventing problematic infections related to IV lines. Moreover, the encasement devices described herein can be used to bundle together lines and/or to secure lines to a patient's bed or other nearby structure, further reducing movement of such lines and potential infection. In some embodiments, the ability to bundle and secure lines can also improve risk of accidental line removal from pediatric patients and/or undesirable entanglement of lines with the patients and/or nearby structure.

In some embodiments, encasement devices described herein can be configured to fold into a compact configuration for storage and/or disposal. For example, an encasement device can be implemented as a sterile, folded up sleeve made of thin, sturdy, anti-microbial plastic. In some embodiments, the sleeve can protect central lines from immediately above the patient's IV ports to the IV pump.

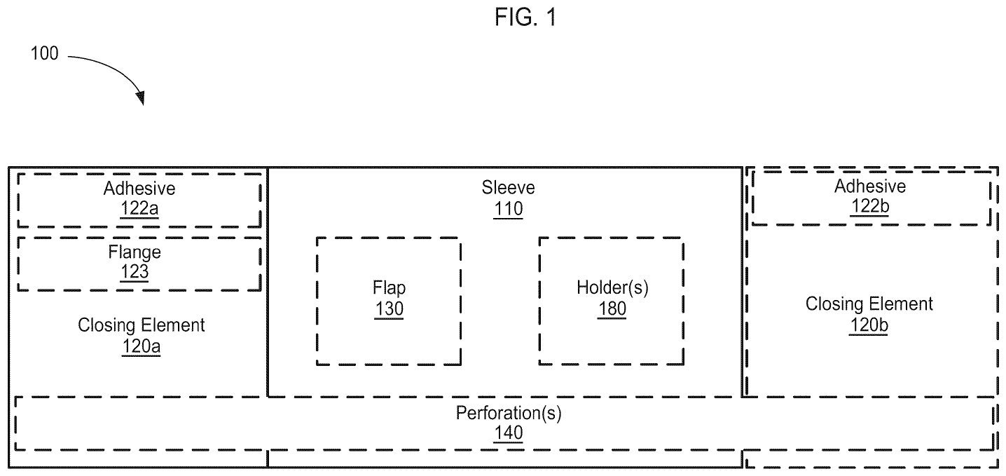

FIG. 1 is a block diagram of a device 100 for protection of one or more central lines from contaminants, according to an embodiment. As shown, the device 100 includes an encasement or sleeve 110 and at least one closing element 120a, 120b (collectively referred to as closing elements 120). In some embodiments, the device 100 can include adhesives 122a, 122b (collectively referred to as adhesives 122) disposed on and/or incorporated into the closing elements 120. In some embodiments, the device 100 can optionally include a flange 123 incorporated into one of the closing elements 120 (e.g., closing element 120a that is situated near a catheter site). In some embodiments, the device 100 can include a flap 130, perforation(s) 140, and/or a holder 180.

In some embodiments, the sleeve 110 can be composed of one or more thermoplastics, including but not limited to: polyvinyl chloride (PVC), polypropylene (PP), polyethylene (PE)—including low-density, linear low-density, medium-density, high-density polyethylene (LDPE, LLDPE, MDPE, HDPE), polystyrene (PS), Nylon, polyethylene terephthalate (PET, PETG), polyimide (PA), polycarbonate (PC), polytetrafluorethylene (PTFE), acrylonitrile butadiene (ABS), polyetheretherketone (PEEK), polyurethane (PU), rubbers, polydimethylsiloxane (PDMS), thermoplastic polyurethanes (TPU), or any other suitable material or combinations thereof.

In some embodiments, the sleeve 110 can be composed of environmentally friendly materials (e.g., compostable and/or biodegradable) materials. Accordingly, in some embodiments, the sleeve 110 can be composed of a bioplastic. In some embodiments, the sleeve 110 can be composed of a naturally, or synthetically, derived biopolymer including but not limited to polylactic acid (PLA), polyhydroxyalkanoates (PHA), Polyhydroxybutyrate (PHB), poly-(3-hydroxybutyrate-co-3-hydroxyhexanoate (PHBH), poly-(3-hydroxybutyrate-co-3-hydroxyvalerate (PHBV), polyvinyl alcohol (PVOH), polybutylene succinate (PBS), rubbers, copolymers, or any other suitable biopolymer or combinations thereof. In some embodiments, the sleeve 110 can be composed of a naturally occurring material, such as starch, cellulose, nanocellulose, lignin, hemicellulose, chitin, chitosan, beta-glucans, glucan, or any other suitable material, or combinations thereof.

In some embodiments, the sleeve 110 can be composed of a blend or mix of one or more of the aforementioned polymers, biopolymers, and/or additives. In some embodiments, the sleeve 110 can be composed of a material to support biodegradation, composting, hydrolysis, and/or rapid degradation in the environment.

In some embodiments, the sleeve 110 can be modified using fillers and/or additives for tuning aesthetic, function, texture, surface smoothness, density, strength, heat-resistance, or other physical characteristics. Such additives may include, but are not limited to: pigments, clarifiers, talc, minerals, calcium carbonates, diatomaceous earths, fire-retardants, fibers, copolymers, stabilizers, lubricants, plasticizers, foaming agents, anti-inflammatory agents, antimicrobials, antifungals, or any combination thereof.

In some embodiments, the sleeve 110 can include antimicrobial additives or antimicrobial agents, e.g., to support efficacy of infection prevention during prolonged use. Such additives can include one or more antimicrobial categories, such as oligodynamic metals (e.g., silver, zinc, copper, nickel, and/or derived alloys including but not limited to brasses, bronzes, cupronickel, copper-nickel-zinc, and/or ionized derivatives), antimicrobial organics including but not limited to organic moieties found in essential oils, ionic polymers and oligomers, ionic rubbers, organometallics, and/or other antimicrobial compounds. In some embodiments, antimicrobial additives can be embedded and distributed within the sleeve 110. In some embodiments, the antimicrobial additives can be coated onto one or many or all external and/or internal surfaces of the sleeve 110. In some embodiments, the antimicrobial additives can coat an inner surface of the sleeve 110. In some embodiments, the antimicrobial additives can coat an outer surface of the sleeve 110. In some embodiments, a first antimicrobial additive can be embedded in the sleeve 110 while a second antimicrobial additive can coat the inner surface and/or the outer surface of the sleeve 110. In some embodiments, one or more antimicrobial additives can be disposed throughout an entire length of the sleeve 110. In some embodiments, the second antimicrobial additive can be different from the first antimicrobial additive. In some embodiments, the second antimicrobial additive can have a different chemical composition from the first antimicrobial additive.

In some embodiments, the sleeve 110 can be opaque. In some embodiments, the sleeve 110 can be at least partially transparent. In some embodiments, the sleeve 110 can have an optical transmittance of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, or at least about 85%. In some embodiments, the sleeve 110 can have an optical transmittance of no more than about 90%, no more than about 85%, no more than about 80%, no more than about 75%, no more than about 70%, no more than about 65%, no more than about 60%, no more than about 55%, no more than about 50%, no more than about 45%, no more than about 40%, no more than about 35%, no more than about 30%, no more than about 25%, no more than about 20%, or no more than about 15%.

Combinations of the above-referenced optical transmittance values are also possible (e.g., at least about 10% and no more than about 90% or at least about 50% and no more than about 70%), inclusive of all values and ranges therebetween. In some embodiments, the sleeve 110 can have an optical transmittance of about 10%, about 15%, about 20%, about 25%, about 30%, about 35%, about 40%, about 45%, about 50%, about 55%, about 60%, about 65%, about 70%, about 75%, about 80%, about 85%, or about 90%.

In some embodiments, the sleeve 110 can include an opaque or substantially opaque label patch disposed thereon. In some embodiments, the opaque label patch can include writing thereon.

In some embodiments, the sleeve 110 can be waterproof, e.g., to protect lines and/or tubing encased or covered by the sleeve 110 from contamination. In some embodiments, the sleeve 110 can be tinted in different colors. In some embodiments, the sleeve 110 can be available in different colors to make the sleeve 110 and the enclosed lines more visible to healthcare staff, thereby reducing the tripping hazard associated with the central lines. In some embodiments, different color sleeves can be used to identify different types of lines, e.g., central or peripheral, and/or uses for different patient populations, e.g., higher or lower risk patient. In some embodiments, the sleeve 110 can be at least partially transparent or translucent, e.g., to make the enclosed lines visible to healthcare staff, thereby facilitating manipulation and/or identification of particular lines.

The sleeve 110 can be sized to receive and enclose a set of one or more medical lines connected to a patient. In some embodiments, the sleeve 110 can enclose at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, or at least about 19 medical lines. In some embodiments, the sleeve 110 can enclose no more than about 20, no more than about 19, no more than about 18, no more than about 17, no more than about 16, no more than about 15, no more than about 14, no more than about 13, no more than about 12, no more than about 11, no more than about 10, no more than about 9, no more than about 8, no more than about 7, no more than about 6, no more than about 5, no more than about 4, no more than about 3, or no more than about 2 medical lines. Combinations of the above-referenced number of medical lines enclosed by the sleeve 110 are also possible (e.g., at least about 1 and no more than about 20 or at least about 2 and no more than about 5), inclusive of all values and ranges therebetween. In some embodiments, the sleeve 110 can enclose about 1, about 2, about 3, about 4, about 5, about 6, about 7, about 8, about 9, about 10, about 11, about 12, about 13, about 14, about 15, about 16, about 17, about 18, about 19, or about 20 medical lines.

The sleeve 110 can be formed from any suitable process for forming a flexible component that is sized to fit around one or more lines or tubing (e.g., central lines or IV lines). In some embodiments, the sleeve 110 can be formed from an extrusion process, such as, for example, a blown film extrusion process. In some embodiments, the sleeve 110 can have a thickness of at least about 100 μm, at least about 200 μm, at least about 300 μm, at least about 400 μm, at least about 500 μm, at least about 600 μm, at least about 700 μm, at least about 800 μm, at least about 900 μm, at least about 1 mm, at least about 1.1 mm, at least about 1.2 mm, at least about 1.3 mm, at least about 1.4 mm, at least about 1.5 mm, at least about 1.6 mm, at least about 1.7 mm, at least about 1.8 mm, or at least about 1.9 mm. In some embodiments, the sleeve 110 can have a thickness of no more than about 2 mm, no more than about 1.9 mm, no more than about 1.8 mm, no more than about 1.7 mm, no more than about 1.6 mm, no more than about 1.5 mm, no more than about 1.4 mm, no more than about 1.3 mm, no more than about 1.2 mm, no more than about 1.1 mm, no more than about 1 mm, no more than about 900 μm, no more than about 800 μm, no more than about 700 μm, no more than about 600 μm, no more than about 500 μm, no more than about 400 μm, no more than about 300 μm, or no more than about 200 μm. Combinations of the above-referenced thickness ranges are also possible (e.g., at least about 100 μm and no more than about 2 mm or at least about 300 μm and no more than about 1 mm), inclusive of all values and ranges therebetween. In some embodiments, the sleeve 110 can have a thickness of about 100 μm, about 200 μm, about 300 μm, about 400 μm, about 500 μm, about 600 μm, about 700 μm, about 800 μm, about 900 μm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, or about 2 mm. In some embodiments, the sleeve 110 can be thin enough, such that it can fit through closed doors.

The sleeve 110 can have a length that is suitable for extending a full length of one or more lines or tubing, e.g., from a catheter site (e.g., a dressing site) to a IV bag, pump, or other external component coupled to a catheter line. In some embodiments, the sleeve 110 can have a length of at least about 1 cm, at least about 2 cm, at least about 3 cm, at least about 4 cm, at least about 5 cm, at least about 6 cm, at least about 7 cm, at least about 8 cm, at least about 9 cm, at least about 10 cm, at least about 20 cm, at least about 30 cm, at least about 40 cm, at least about 50 cm, at least about 60 cm, at least about 70 cm, at least about 80 cm, at least about 90 cm, at least about 1 m, at least about 2 m, at least about 3 m, at least about 4 m, at least about 5 m, at least about 6 m, at least about 7 m, at least about 8 m, or at least about 9 m. In some embodiments, the sleeve 110 can have a length of no more than about 10 m, no more than about 9 m, no more than about 8 m, no more than about 7 m, no more than about 6 m, no more than about 5 m, no more than about 4 m, no more than about 3 m, no more than about 2 m, no more than about 1 m, no more than about 90 cm, no more than about 80 cm, no more than about 70 cm, no more than about 60 cm, no more than about 50 cm, no more than about 40 cm, no more than about 30 cm, no more than about 20 cm, no more than about 10 cm, no more than about 9 cm, no more than about 8 cm, no more than about 7 cm, no more than about 6 cm, no more than about 5 cm, no more than about 4 cm, no more than about 3 cm, or no more than about 2 cm.

Combinations of the above-referenced lengths of the sleeve 110 are also possible (e.g., at least about 1 cm and no more than about 10 mm or at least about 5 cm and no more than about 50 cm), inclusive of all values and ranges therebetween. In some embodiments, the sleeve 110 can have a length of about 1 cm, about 2 cm, about 3 cm, about 4 cm, about 5 cm, about 6 cm, about 7 cm, about 8 cm, about 9 cm, about 10 cm, about 20 cm, about 30 cm, about 40 cm, about 50 cm, about 60 cm, about 70 cm, about 80 cm, about 90 cm, about 1 m, about 2 m, about 3 m, about 4 m, about 5 m, about 6 m, about 7 m, about 8 m, about 9 m, or about 10 m.

In some embodiments, the sleeve 110 can include one or more coupling elements (not shown) disposed along a length of the sleeve 110. The coupling element can be configured to couple or attach one or more portions of the sleeve 110 to nearby structures (e.g., a patient bed, an IV pole, walls, furniture, medical equipment, etc.), e.g., to secure lines and sleeves for safety and convenience. In an embodiment, the coupling elements can be implemented as mounting loops or straps. The mounting loops can include elastic elements (e.g., elastic string) that are attached to adhesive strips or pads each with a removable mounting sticker. The stickers can be removed to expose adhesive portions that can be used to secure or tether a portion of the sleeve to an object. Additionally, or alternatively, one or more coupling elements can include other structure for attaching to objects, e.g., hooks, fasteners, clamps, etc. While the sleeve 110 can protect lines and other tubing from potential exposure to pathogens, it can also be used to protect sensitive lines and/or lines that may be exposed to the floor or other environments where damage to the lines may be possible. Additionally or alternatively, the coupling elements can allow healthcare workers to keep the sleeve 110 and catheter lines out of certain areas, e.g., so they pose less of a hazard for tripping, which could cause injury to the healthcare professional or the patient. In some embodiments, the sleeve 110 can be discarded and replaced at the same time one or more lines are exchanged or removed.

The closing elements 120 can have one or more mechanisms to close terminal ends of the sleeve 110. The closing elements 120 can be configured to transition the terminal ends of the sleeve 110 between a closed or sealed configuration and an open configuration. In the open configuration, the terminal ends of the sleeve 110 can permit passage of one or more catheter lines. In the closed or sealed configuration, the terminal ends of the sleeve 110 can close around one or more catheter sites or lines, e.g., securing the sleeve 110 in place and enhancing the effectiveness of the barrier created by the sleeve 110. In some embodiments, closure types and mechanisms employed in the closing elements 120 can include a drawstring and/or a cinch. In some embodiments, the drawstring and/or cinch can include a cord. In some embodiments, the drawstring and/or cinch can be braided. In some embodiments, the drawstring and/or cinch can be composed of plastic, rubber, polymer, or any other suitable material or combinations thereof. In some embodiments, the drawstring and/or cinch can include stopper snaps disposed thereon. In some embodiments, the stopper snaps can include round buttons (e.g., snap fit buttons) disposed on an elastic drawstring that can couple to one another to tighten the sleeve 110 around the catheter lines and keep the sleeve 110 in place. In some embodiments, the stopper snaps can be made of a rubbery material and can slide along the length of an elastic cord to tighten the sleeve 110 and secure the sleeve 110 in place. In some embodiments, one or more elastic cords of the closing elements 120 can be disposed in a channel, fold, or seam at either end of the sleeve 110. In some embodiments, the elastic cords can include a knotted end that can be used to secure the sleeve 110 to nearby surfaces. In some embodiments, the channel in which the closing elements 120 are disposed can be made by folding over the material of the sleeve 110 and securing it in place (e.g., via an adhesive and/or stitching). After forming the channel, the elastic cords can be extended through the channel and knotted. In use, a healthcare worker can slide the stopper snaps along the elastic cord to tighten the sleeve around the central lines and snap them closed to keep the sleeve 110 secured around the lines. In some embodiments, a portion of the knotted end of the elastic cord can be used to affix or secure the sleeve 110 to an appropriate surface or object.

In some embodiments, the closing elements 120 can optionally include adhesives 122. In some embodiments, the adhesives 122 can include self-adhesives, two-sided adhesives, one-sided adhesives, flap-hinged adhesives, resealable adhesives, or any other suitable adhesives or combinations thereof. In some embodiments, at least one of the closing elements 120 (e.g., closing element 120a) can include a flange 123. In some embodiments, the flange can be an adhesive flange, which can be a peel strength adhesive flange, an angle adhesive flange, or any other suitable adhesive flange or combinations thereof. The flanges 123 can provide a surface for adhering to a medical dressing. The flanges 123 can adhere to a medical dressing overlaying an insertion site. In some embodiments, the flanges 123 can encircle the insertion site. In some embodiments, the adhesive 122a and/or the adhesive 122b can adhere to itself and/or an inner or outer surface of the sleeve 110 to close around the central line. In some embodiments, the closing element 120a and/or the closing element 120b can be configured to selectively open and close the terminal ends of the sleeve 110. In some embodiments, the adhesive 122a and/or the adhesive 122b can adhere to close around the central line without adhering to a dressing. In some embodiments, the adhesives 122 can include an adhesive pad or strip that can selectively open and close multiple times. In some embodiments, the flange 123 can have a rectangular shape, a square shape, a round shape, an annular shape, a trigonal shape, a hexagonal shape, an octagonal shape, or any other suitable shape.

In some embodiments, the adhesive flange can have a peel strength of at least about 1 N/25 mm, at least about 2 N/25 mm, at least about 3 N/25 mm, at least about 4 N/25 mm, at least about 5 N/25 mm, at least about 6 N/25 mm, at least about 7 N/25 mm, at least about 8 N/25 mm, at least about 9 N/25 mm, at least about 10 N/25 mm, at least about 12 N/25 mm, at least about 14 N/25 mm, at least about 15 N/25 mm, at least about 16 N/25 mm, at least about 18 N/25 mm, at least about 20 N/25 mm, at least about 25 N/25 mm, at least about 30 N/25 mm, at least about 35 N/25 mm, at least about 40 N/25 mm, or at least about 45 N/25 mm. In some embodiments, the adhesive flange can have a peel strength of no more than about 50 N/25 mm, no more than about 45 N/25 mm, no more than about 40 N/25 mm, no more than about 35 N/25 mm, no more than about 30 N/25 mm, no more than about 25 N/25 mm, no more than about 20 N/25 mm, no more than about 18 N/25 mm, no more than about 16 N/25 mm, no more than about 15 N/25 mm, no more than about 14 N/25 mm, no more than about 12 N/25 mm, no more than about 10 N/25 mm, no more than about 9 N/25 mm, no more than about 8 N/25 mm, no more than about 7 N/25 mm, no more than about 6 N/25 mm, no more than about 5 N/25 mm, no more than about 4 N/25 mm, no more than about 3 N/25 mm, or no more than about 2 N/25 mm.

Combinations of the above-referenced peel strengths are also possible (e.g., at least about 1 N/25 mm and no more than about 50 N/25 mm or at least about 5 N/25 mm and no more than about 25 N/25 mm), inclusive of all values and ranges therebetween. In some embodiments, the adhesive flange can have a peel strength of about 1 N/25 mm, about 2 N/25 mm, about 3 N/25 mm, about 4 N/25 mm, about 5 N/25 mm, about 6 N/25 mm, about 7 N/25 mm, about 8 N/25 mm, about 9 N/25 mm, about 10 N/25 mm, about 12 N/25 mm, about 14 N/25 mm, about 15 N/25 mm, about 16 N/25 mm, about 18 N/25 mm, about 20 N/25 mm, about 25 N/25 mm, about 30 N/25 mm, about 35 N/25 mm, about 40 N/25 mm, about 45 N/25 mm, or about 50 N/25 mm.

In some embodiments, the closing elements 120 can include hook and loop closing elements, self-fasteners, self-closures, zip top closures, snap-button closures, press-to-seal closures, snap closures, Velcro closures, or any other suitable closures or combinations thereof. In some embodiments, the closing element 120a can include a first closing mechanism and the closing element 120b can include a second closing mechanism, the second closing mechanism different from the first closing mechanism. For example, the closing element 120a can include an adhesive flange, while the closing element 120b can include a drawstring closure.

While sleeve 110 is depicted with two closing elements 120a, 120b, e.g., disposed at the opposite ends of the sleeve 110, it can be appreciated that any number of closing elements 120a, 120b can be part of device 100. For example, in some embodiments, a sleeve 110 can have one end that can open and close and an opposite end that is permanently closed (e.g., sealed). In such embodiments, the sleeve 110 may only have a single closing element 120a. In other words, the sleeve 110 can effectively act as a bag with one open end and one closed end. In other embodiments, the sleeve 110 can have multiple closing elements 120 disposed along a length of the sleeve 110, e.g., to secure the sleeve 110 around internal lines at multiple points and/or to secure the sleeve 110 to nearby surfaces or objects at multiple points. In some embodiments, the closing elements 120 can include stopper snaps to secure the closing elements 120 in place.

In some embodiments, an adhesive flange incorporated into one or more of the closing elements 120 can be configured to attach to various surfaces (e.g., medical dressings, patient skin, etc.). In some embodiments, an adhesive flange that is attached to a particular surface can be designed to be removed at a low peel strength, e.g., to prevent disruption of an existing dressing. In some embodiments, the sleeve 110 can be discarded after a single removal. In some embodiments, the adhesive flange can have adhesive properties and peel strength for repeated manipulation, adjustment, or placement, such that the encasement may be accessed repeatedly throughout use, not disrupt the foundation dressing, and retain effective adhesion strength through multiple uses. In such embodiments, the adhesive flange can be replaced on the dressing one or more times, e.g., forming effective barrier properties for continued use until discarded. In some embodiments, the adhesive forming the seal of the adhesive flange can be embedded and/or coated with antimicrobial additives.

In some embodiments, the sleeve 110 can include a higher concentration of antimicrobial additives at the terminal ends of the sleeve 110 than near the center of the sleeve 110.

In some embodiments, a cinch style closure method such as a drawstring or cord may be used at the end of the sleeve 110 to close the sleeve 110 firmly around one or more catheter lines. In some embodiments, the drawstring/cord of the cinching mechanism incorporated into the closing elements 120 can be composed of a synthetic material, e.g., to prevent liquid absorption. In some embodiments, the drawstring/cord of the cinching mechanism incorporated into the closing elements 120 can include antimicrobial materials. In some embodiments, the drawstring/cord of the cinching mechanism incorporated into the closing elements 120 can include end-tabs for dexterity and/or fray prevention. In some embodiments, seams or folds can be formed, e.g., to envelop one or more components of the closing elements 120 (e.g., the cords/cinch tool, cord, etc.), using techniques such as flange roll (e.g., sleeve 110 being wrapped around a cinching mechanism or portion thereof) and then sealed using heat, impulse sealing, sonic sealing, or other suitable techniques.

In some embodiments, the sleeve 110 can optionally include a flap 130 that can allow for access to the catheter lines within the sleeve 110 without removal of the sleeve 110 and/or largescale manipulation of the sleeve 110 (e.g., scrunching of the sleeve to expose a particular part of a catheter line). The flap 130 can be positioned at a location along the sleeve 110 that access may be more often desired, e.g., a location with connector(s) and/or port(s) for connecting to other lines, ports, catheters, etc. In some embodiments, the flap 130 can include a section that is integrally formed or coupled to the sleeve 110 with a resealable surface (e.g., an adhesive surface), such that the flap 130 can be opened and closed to maintain the protective qualities of the sleeve 110 after accessing the central lines. In some embodiments, the flap 130 can include a tab attached thereto to ease pulling of the flap 130. In some embodiments, the flap 130 can have the same or substantially similar material properties to the rest of the sleeve 110. In some embodiments, the flap can have different material properties from the rest of the sleeve 110. While a single flap 130 is depicted in FIG. 1, it can be appreciated that devices described herein can include multiple flaps disposed at different locations along a longitudinal length of the sleeve 110. In some embodiments, the flap 130 can include a higher concentration of antimicrobial additives than the remainder of the sleeve 110. In some embodiments, the flap 130 can include a first portion that is attached to or integrally formed with the rest of the sleeve 110 and a second portion having one or more resealable surfaces that can couple to an exterior surface of the rest of the sleeve 110. In other words, a resealable surface on the outside of the flap 130 can reversibly couple to the outer surface of the sleeve 110 when the flap 130 is opened, in order to keep the flap 130 opened securely.

In some embodiments, the sleeve 110 can optionally include the perforation 140. The perforation 140 can allow for easy and/or immediate removal of the sleeve 110. The perforation 140 can allow the sleeve 110 to be opened along the longitudinal length of the sleeve 110 such that the sleeve 110 can be removed from the set of medical lines. In some embodiments, the perforation 140 can extend along the length of the sleeve 110. In some embodiments, the perforation 140 can extend approximately from the closing element 120a to the closing element 120b. In some embodiments, the sleeve 110 can include multiple lines of perforations 140, e.g., a first line that extends along a longitudinal length of the sleeve 110 and one or more other lines that extend around a perimeter of the sleeve 110 (e.g., circumferentially around the sleeve 110). In some embodiments, the perforation 140 can include multiple perforation lines along the length of the sleeve 110. In some embodiments, the perforation 140 can include multiple perforation lines around the perimeter of the sleeve 110. In some embodiments, one or more perforations 140 can be configured to separate portions of the sleeve 110 from one another, e.g., to separate a closing element 120 from an end of the sleeve 110.

In some embodiments, the device 100 can optionally include a holder 180. The holder 180 can be incorporated into and/or disposed within the sleeve 110, e.g., to aid in spacing and organizing the catheter lines. In some embodiments, the holder 180 can include a mechanical coupler. In some embodiments, the holder 180 can include an adhesive. In some embodiments, the holder 180 can extend between the sleeve 110 and a nearby structure or surface and couple the sleeve to the nearby structure or surface. In some embodiments, the holder 180 can include multiple notches, into which the catheter lines can be secured. In some embodiments, the holder 180 can be attached to the interior of the sleeve 110. In some embodiments, the holder 180 can be detached from the sleeve 110 such that the holder 180 is separate from the sleeve 110 and can move relative to the sleeve 110. In some embodiments, the holder 180 can be attached to the interior of the sleeve 110 (e.g., via an adhesive). In some embodiments, the holder can be attached to the exterior of the sleeve 110 (e.g., via an adhesive). In some embodiments, the holder 180 can feature a row of slots inside the sleeve 110 for placement of the catheter lines. In some embodiments, the holder 180 can be located about 1 cm, about 2 cm, about 3 cm, about 4 cm, about 5 cm, about 6 cm, about 7 cm, about 8 cm, about 9 cm, or about 10 cm from the proximal end of the sleeve 110, inclusive of all values and ranges therebetween. In some embodiments, a first end of the encasement 100 can include multiple holders 180 to properly space and organize the catheter lines.

In some embodiments, the holder 180 can be disposed near the flap 130 (e.g., less than about 10 cm, less than about 9 cm, less than about 8 cm, less than about 7 cm, less than about 6cm, less than about 5 cm, less than about 4 cm, less than about 3 cm, less than about 2 cm, or less than about 1 cm from an outside edge of the flap 130, inclusive of all values and ranges therebetween). In some embodiments, the holder 180 can be disposed near an opening created by the lifting of the flap 130 (e.g., less than about 10 cm, less than about 9 cm, less than about 8 cm, less than about 7 cm, less than about 6 cm, less than about 5 cm, less than about 4 cm, less than about 3 cm, less than about 2 cm, or less than about 1 cm from the opening created by the lifting of the flap 130, inclusive of all values and ranges therebetween).

In some embodiments, a designated opaque area or plaque can be printed or layered on the sleeve 110, enabling clear labeling, dating, or annotating using writing utensils such as pens, markers, and/or similar items. In some embodiments, multiple devices 100 can be packaged into a package for ease of access and dispensation of the devices 100 in medical and/or home environments. In some embodiments, the device 100 can be folded in such a way to make storage and usage convenient. In some embodiments, the sleeve 110 can be folded inside itself to make it smaller, e.g., so it can be handled easily by healthcare staff.

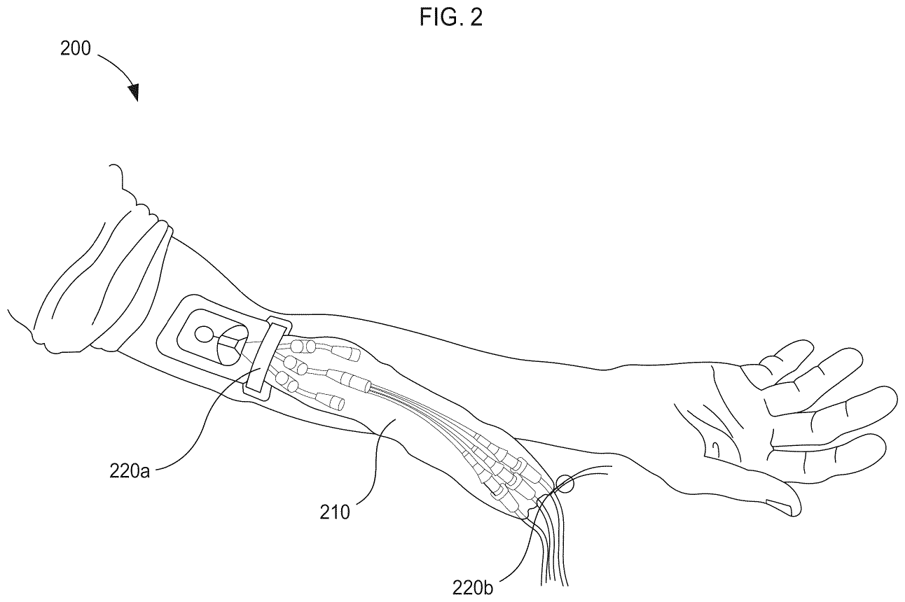

FIG. 2 is an illustration of an encasement device 200 for protection of one or more lines or tubing (e.g., central lines) from contaminants, according to an embodiment. As shown, the encasement device 200 includes an encasement or sleeve 210, a proximal closing element 220a, and a distal closing element 220b (collectively referred to as closing elements 220). In some embodiments, the sleeve 210 and the closing elements 220 can be the same or substantially similar to the sleeve 110 and the closing elements 120, as described above with reference to FIG. 1. Thus, certain aspects of the sleeve 210 and the closing elements 220 are not described in greater detail herein.

As shown, the sleeve 210 covers access ports and other areas of central lines susceptible to contamination. As shown, the proximal closing element 220a is coupled to the dressing via a strap or strip. In some embodiments, the closing element 220a can include a resealable adhesive. The resealable adhesive can be configured to selectively open and close the open end of the sleeve 210, e.g., by adhering to itself and/or an inner or outer surface of the sleeve 210. As shown, the distal closing element 220b secures over and around the lines via a drawstring apparatus. The drawstring apparatus includes a stopper that can move to tighten the distal closing element 220b. In some embodiments, the stopper can include a button that can be pushed against a spring mechanism to allow the stopper to be moved along the drawstring to tighten and loosen the drawstring. In some embodiments, the strap of the proximal closing element 220a can include an adhesive. In some embodiments, the strap of the proximal closing element 220 can seal an end of the sleeve 210.

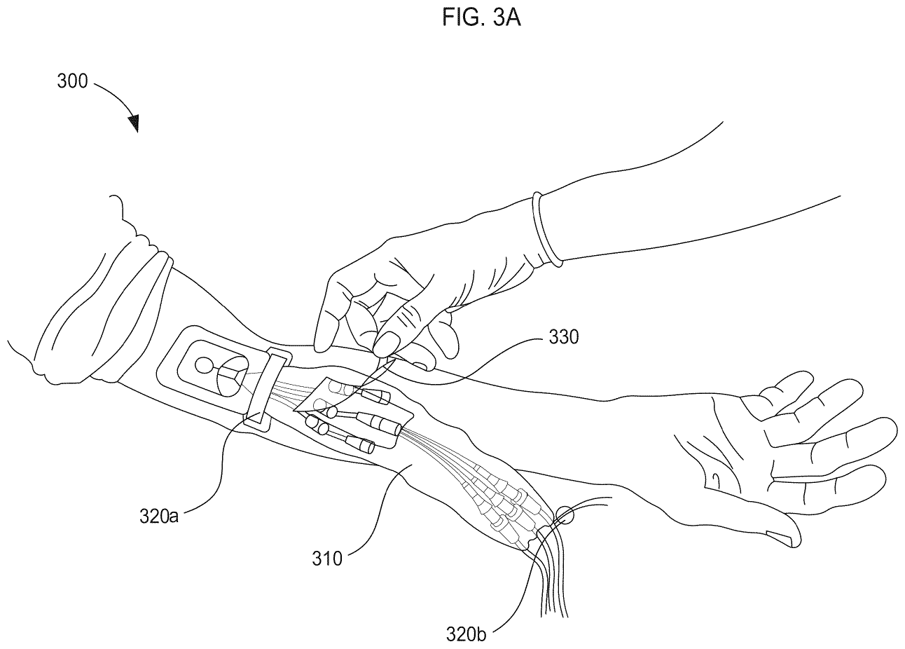

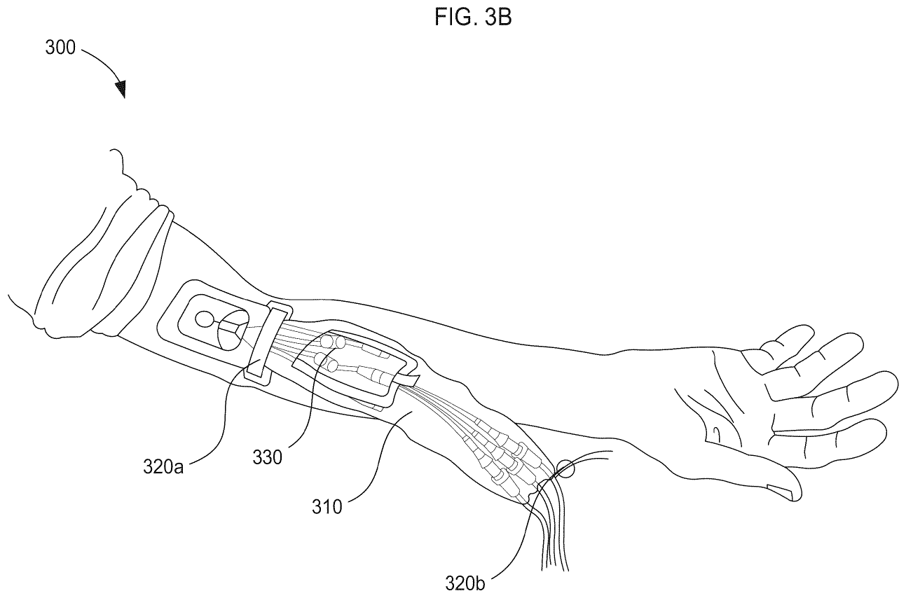

FIGS. 3A and 3B are illustrations of an encasement device 300 with a flap for protection of one or more lines or tubing (e.g., central lines) from contaminants, according to an embodiment. As shown, the encasement device 300 includes a sleeve 310 with a proximal closing element 320a, a distal closing element 320b (collectively referred to as closing elements 320), and a flap 330. As shown, the flap 330 includes a tab 331. In some embodiments, the sleeve 310, the proximal closing element 320a, and the distal closing element 320b can be the same or substantially similar to the sleeve 210, the proximal closing element 220a, and the distal closing element 220b, as described above with reference to FIG. 2. Thus, certain aspects of the sleeve 310, the proximal closing element 320a, and the distal closing element 320b are not described in greater detail herein.

The flap 330 can provide access to the lines and/or connectors along the lines (e.g., ports, Luer connectors, etc.) without removing the sleeve 310. The flap 330 is in an open position as shown in FIG. 3A and a closed position as shown in FIG. 3B. As shown, the tab 331 allows for ease of opening of the flap 330. In some embodiments, the flap 330 can include a resealable surface on the underside of the flap 330. In some embodiments, the resealable surface on the underside of the flap 330 can adhere to the outer surface of the sleeve 310. In some embodiments, the outer surface of the sleeve 310 can include a resealable surface to mate with the resealable surface on the underside of the flap 330. In some embodiments, the flap 330 can have a larger area than the area of the opening created by the removal of the flap 330, such that the flap 330 can cover more than the area of the opening. In some embodiments, the flap 330 can have length (i.e., a dimension in the longitudinal direction of the sleeve 310) of about 1 cm, about 2 cm, about 3 cm, about 4 cm, about 5 cm, about 6 cm, about 7 cm, about 8 cm, about 9 cm, about 10 cm, about 15 cm, about 20 cm, about 25 cm, or about 30 cm, inclusive of all values and ranges therebetween. In some embodiments, the flap 330 can have a width (e.g., a dimension in the lateral direction of the sleeve 310) of about 1 cm, about 2 cm, about 3 cm, about 4 cm, about 5 cm, about 6 cm, about 7 cm, about 8 cm, about 9 cm, or about 10 cm, inclusive of all values and ranges therebetween.

In some embodiments, the flap 330 can be disposed, such that the proximal end of the flap 330 is at least about 1 cm, at least about 2 cm, at least about 3 cm, at least about 4 cm, at least about 5 cm, at least about 6 cm, at least about 7 cm, at least about 8 cm, at least about 9 cm, at least about 10 cm, at least about 15 cm, at least about 20 cm, or at least about 25 cm from the proximal closing element 320a. In some embodiments, the flap 330 can be disposed, such that the proximal end of the flap 330 is no more than about 30 cm, no more than about 25 cm, no more than about 20 cm, no more than about 15 cm, no more than about 10 cm, no more than about 9 cm, no more than about 8 cm, no more than about 8 cm, no more than about 8 cm, no more than about 8 cm, no more than about 7 cm, no more than about 6 cm, no more than about 5 cm, no more than about 4 cm, no more than about 3 cm, or no more than about 2 cm from the proximal closing element 320a. Combinations of the above-referenced distances are also possible (e.g., at least about 1 cm and no more than about 30 cm or at least about 3 cm and no more than about 10 cm), inclusive of all values and ranges therebetween. In some embodiments, the flap 330 can be disposed, such that the proximal end of the flap 330 is about 1 cm, about 2 cm, about 3 cm, about 4 cm, about 5 cm, about 6 cm, about 7 cm, about 8 cm, about 9 cm, about 10 cm, about 15 cm, about 20 cm, about 25 cm, or about 30 cm from the proximal closing element 320a.

In some embodiments, the tab 331 can have a length suitable for a healthcare worker or a patient to hold by hand (e.g., via a thumb and index finger) and pull away to access the central lines. In some embodiments, the tab 331 can have a length of about 1 mm, about 2 mm, about 3 mm, about 4 mm, about 5 mm, about 6 mm, about 7 mm, about 8 mm, about 9 mm, about 1 cm, about 2 cm, about 3 cm, about 4 cm, about 5 cm, about 6 cm, about 7 cm, about 8 cm, about 9 cm, or about 10 cm, inclusive of all values and ranges therebetween. A tab may be attached to any or all layers of the device.

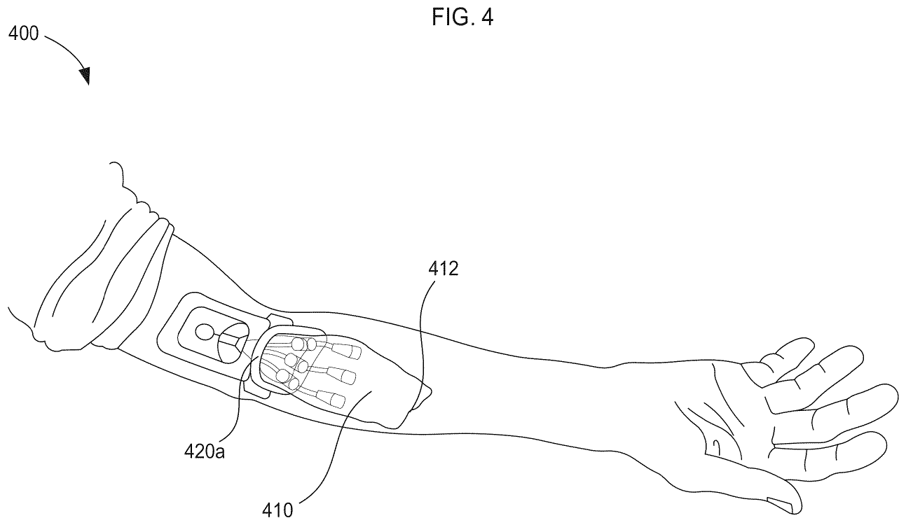

FIG. 4 is an illustration of an encasement device 400 for protection of one or more lines or tubing (e.g., central lines) from contaminants, according to an embodiment. As shown, the encasement device 400 includes a sleeve 410 with a distal closed end 412 and a proximal closing element 420a. In some embodiments, the sleeve 410 and the proximal closing element 420a can be the same or substantially similar to the sleeve 210 and the closing element 220a, as described above with reference to FIG. 2. Thus, certain aspects of the sleeve 410 and the proximal closing element 420a are not described in greater detail herein.

As shown, the closed end 412 is permanently closed. In other words, the sleeve 410 is configured as a bag, with one open end and one closed end. The sleeve 410 with the closed end 412 can be of particular use when the patient is not receiving treatment via the lines but still needs to be protected from contamination between treatments.

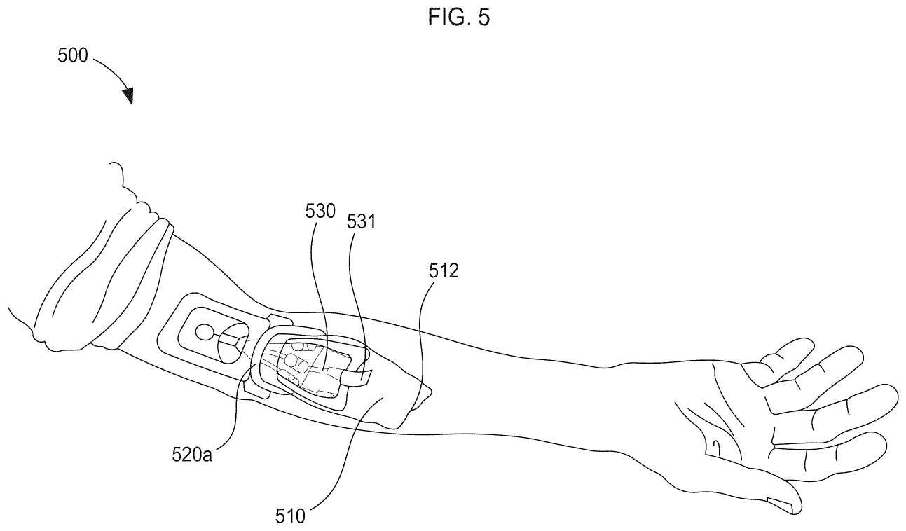

FIG. 5 is an illustration of an encasement device 500 for protection of one or more lines or tubing (e.g., central lines) from contaminants, according to an embodiment. As shown, the encasement device 500 includes a sleeve 510 with a distal closed end 512 and a proximal closing element 520a, as well as a flap 530. In some embodiments, the sleeve 510, the closed end 512, and the proximal closing element 520a can be the same or substantially similar to the sleeve 410, the closed end 412, and the proximal closing element 420a, as described above with reference to FIG. 4. In some embodiments, the flap 530 and the tab 531 can be the same or substantially similar to the flap 330 and the tab 331, as described above with reference to FIGS. 3A and 3B. Thus, certain aspects of the sleeve 510, the terminal end 512, the proximal closing element 520a, the flap 530, and the tab 531 are not described in greater detail herein.

Inclusion of the closed end 512 with the window 530, and tab 531 can reduce the risk of exposure to biofilm or contaminates on the central lines and ports during non-use while allowing access to the central lines and ports. In some embodiments, intermittent IV fluid infusion, hemodialysis treatment, or intravenous push of medication can be given via an entrance through the opening of the sleeve window 530 via the tab 531.

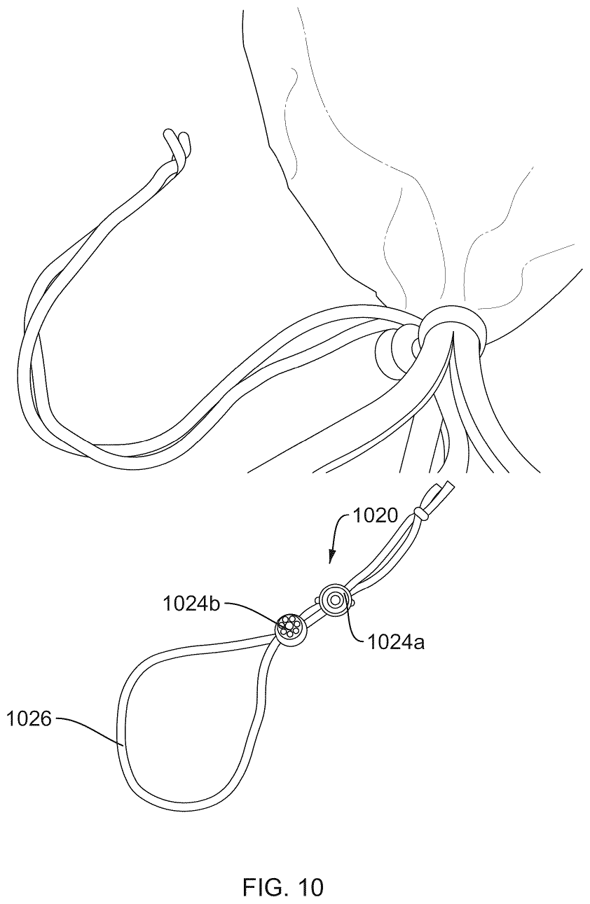

FIG. 10 is an illustration of an example closing element 1020, and its implementation in a sleeve, according to an embodiment. As shown, the closing element 1020 includes stopper snaps 1024a, 1024b (collectively referred to as stopper snaps 1024) and an elastic cord 1026. In some embodiments, the closing element 1020 can be the same or substantially similar to the closing element 120, as described above with reference to FIG. 1. Thus, certain aspects of the closing element 1020 are not described in greater detail herein.

As shown, the closing element 1020 includes two stopper snaps 1024. In some embodiments, the stopper snap 1024a can provide an anchor while the stopper snap 1024b can move along the elastic cord 1026 to tighten and loosen the grip of the elastic cord 1026 over the central lines. In some embodiments, the elastic cord 1026 can be partially disposed in a tunnel or channel at a terminal end of the sleeve during use. In some embodiments, the closing element 1020 can include 1, 2, 3, 4, 5, 6, 7, 8, 9, 10, or at least about 10 stopper snaps 1024, inclusive of all values and ranges therebetween.

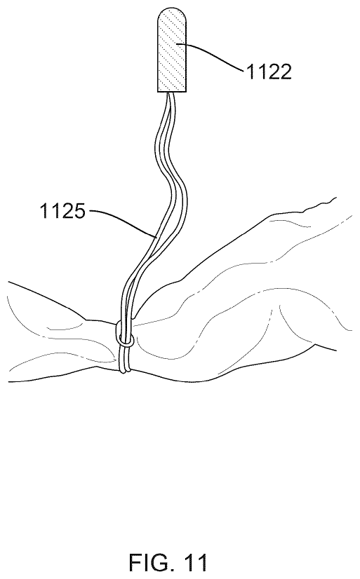

FIG. 11 is an illustration of an example mounting loop 1125 of an encasement device, according to an embodiment. As shown, the mounting loop 1125 includes a mounting strip 1122. The mounting strip 1122 can attach a terminal end of the mounting loop 1125 to a desired location (e.g., walls, furniture, patient bed, etc.). In some embodiments, the mounting strip 1122 can include an adhesive surface disposed thereon. In some embodiments, a strip of material can be removed from the mounting strip 1122 to expose the adhesive surface. In some embodiments, the adhesive surface of the mounting strip 1122 can be reusable, such that the mounting strip 1122 can be removed and re-attached. In some embodiments, the mounting loop 1125 can be incorporated into a closing element (e.g., closing element 120 or any of the other closing elements described herein). In other words, a closing element can close an end of the sleeve with the mounting strip 1122 attached to a portion (e.g., terminal end) of the closing element.

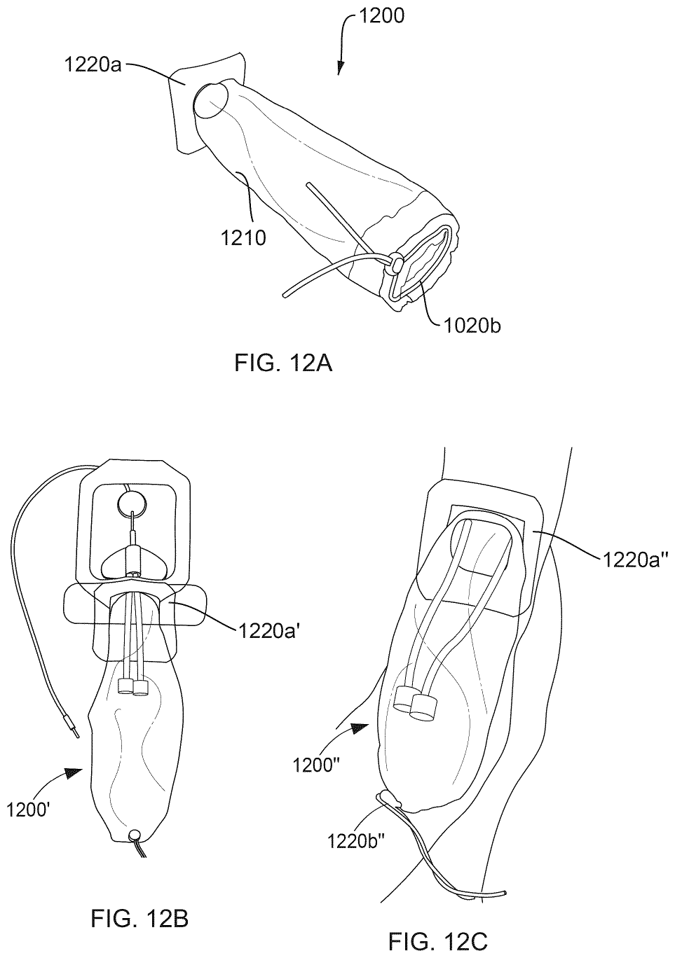

FIGS. 12A-12C are illustrations of encasement devices for protection of one or more lines or tubing (e.g., central lines) from contaminants, according to various embodiments. FIG. 12A shows an encasement device 1200 with a sleeve 1210 and closing elements 1220a, 1220b. As shown, the closing element 1220a includes an adhesive flange for coupling to a dressing, while the closing element 1220b includes a drawstring or cinch. FIG. 12B shows an encasement device 1200′ mounted to a wall to show details of the dressing and the central lines running through the encasement device 1200′. The encasement device 1200′ can include a closing element 1220a′ including an adhesive strip and/or flange configured to secure to the dressing. FIG. 12C shows an encasement device 1200″ mounted directly to a patient's arm, with a closing element 1220a′ at a proximal end implemented as a flange and a closing element 1220b′ at a distal end of the encasement in a closed position (and the central lines contained within the encasement device 1200″).

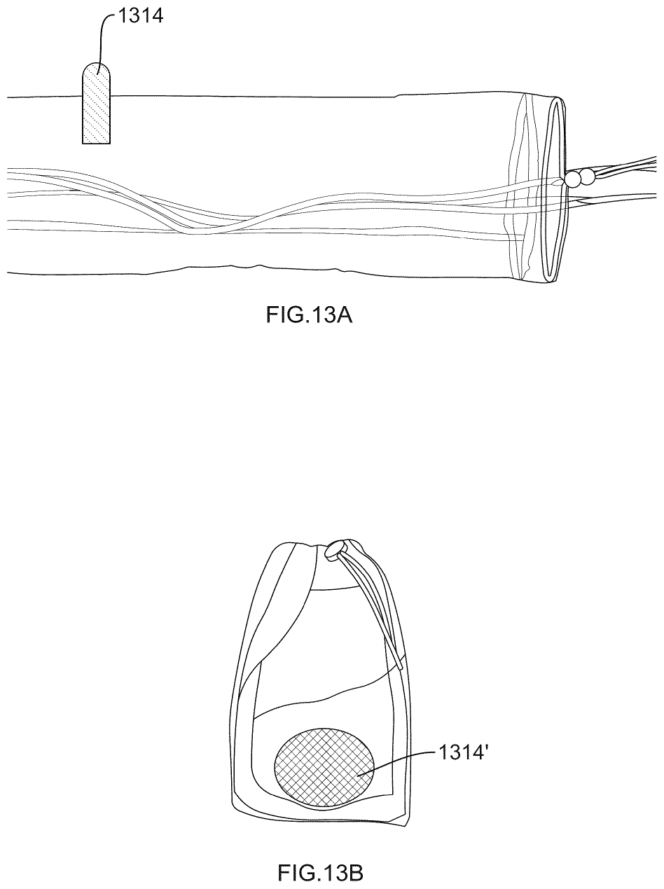

FIGS. 13A and 13B are illustrations of encasement devices with adhesive elements for securement to surfaces, according to various embodiments. The adhesive elements can be examples of coupling elements, as described with reference to FIG. 1. As shown, the encasement device in FIG. 13A includes an encasement or sleeve with an adhesive strip 1314 coupled directly to the surface of the sleeve. The adhesive strip 1314 can allow the sleeve to be coupled directly to a desired location (e.g., walls, furniture, patient bed, etc.). In some embodiments, the adhesive strip 1314 can be reusable such that it can be re-applied after becoming detached from a surface. In some embodiments, the adhesive strip 1314 can include a portion that can be removed to expose the adhesive surface. FIG. 13B is an illustration of an encasement with an adhesive element 1314′ coupled directly to the sleeve. As shown, the adhesive element 1314′ has a round shape. In some embodiments, the adhesive element 1314′ can include a portion that can be removed to expose the adhesive surface.

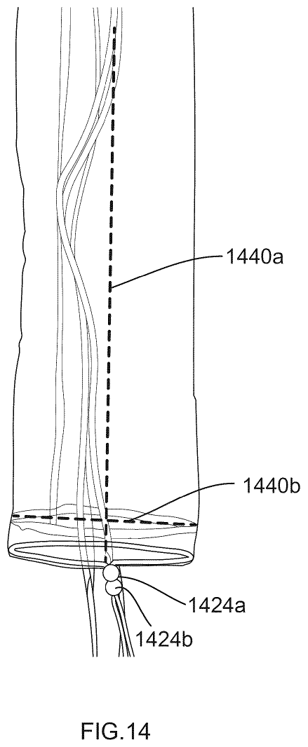

FIG. 14 is an illustration of an encasement device with perforations, according to an embodiment. As shown, the encasement device includes a longitudinal perforation 1440a and a circumferential perforation 1440b. The perforations 1440a, 1440b and other components of the encasement device as depicted in FIG. 14 can be structurally and/or functionally similar to other perforations (e.g., perforation 140) and components, as described herein. The longitudinal perforation 1440a extends to at least one end (e.g., the proximal end) of the sleeve. In some embodiments, the longitudinal perforation 1440a extends the entire length of the sleeve, from one terminal end to another terminal end, so the sleeve can be quickly opened and/or removed. In some embodiments, the longitudinal perforation 1440a can extend to a location near the proximal end of the sleeve, e.g., distal to it by about 1 cm, about 2 cm, about 3 cm, about 4 cm, about 5 cm, about 6 cm, about 7 cm, about 8 cm, about 9 cm, about 10 cm, inclusive of all values and ranges therebetween. As shown, the circumferential perforation 1440b can extend around the entire circumference of the sleeve. In some embodiments, the circumferential perforation 1440b can extend around about 40%, about 50%, about 60%, about 70%, about 80%, or about 90% of the circumference of the sleeve, inclusive of all values and ranges therebetween. In use, a healthcare professional can open the sleeve along the longitudinal perforation 1440a and/or detach a closing element including stopper snaps 1424a, 1424b from the sleeve via circumferential perforation 1440b. Removal of the closing element from the sleeve and perforating along a length of the sleeve can enable a user to remove the sleeve from being around one or more lines that are within a patient.

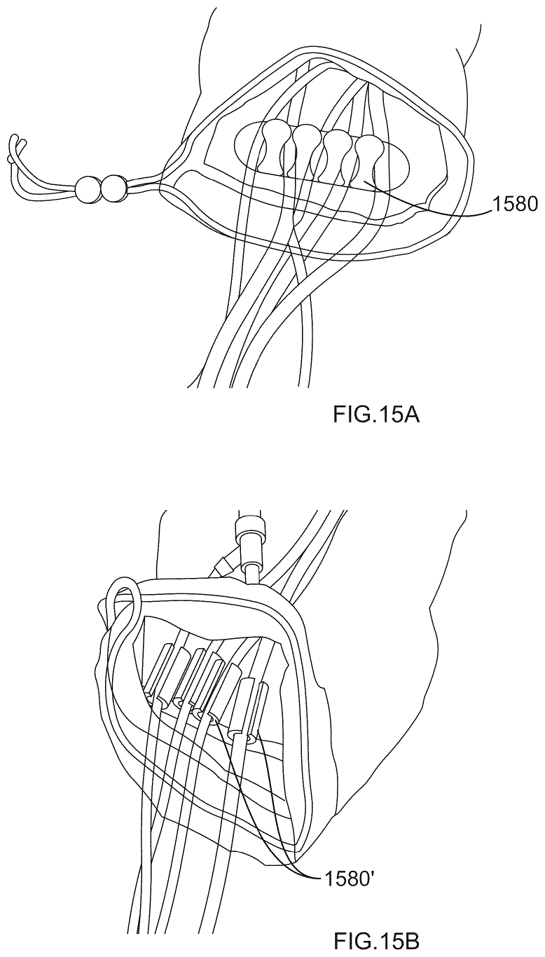

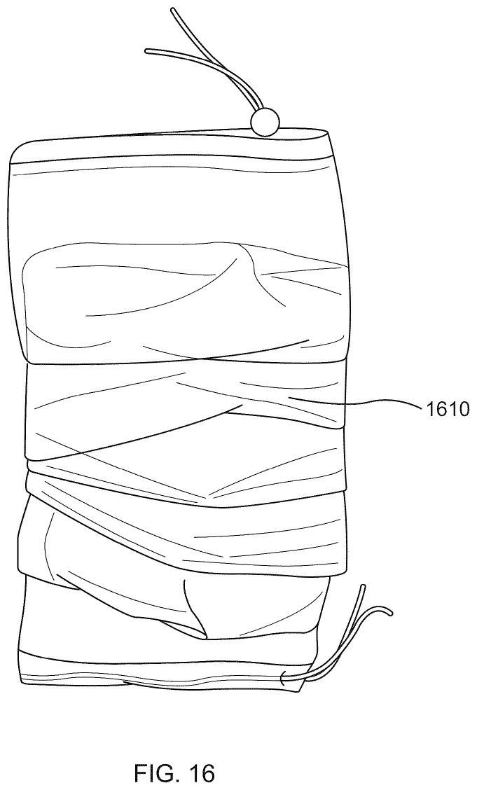

FIGS. 15A and 15B are illustrations of encasement devices with holders, according to various embodiments. FIG. 15A includes a holder 1580 with five IV tubes secured therein. In other words, the holder 1580 includes securements for five IV tubes. In some embodiments, the holder 1580 can include securements for 1, 2, 3, 4, 6, 7, 8, 9, 10, or more than about 10 IV tubes. As shown, the holder 1580 includes rounded cavities for insertion and securement of central lines. In some embodiments, the holder 1580 can be secured directly to the interior of the sleeve. In some embodiments, the holder 1580 can be detached from the interior of the sleeve. FIG. 15B includes a holder 1580′ with four IV tubes secured into clips on the holder 1580.′ In some embodiments, the holder 1500′ can be attached to the interior of the sleeve. In some embodiments, the holder 1580′ can be detached from the interior of the sleeve. The holders 1580, 1580′ can be structurally and/or functionally similar to other holders described herein, including holder(s) 180 as described with respect to FIG. 1.

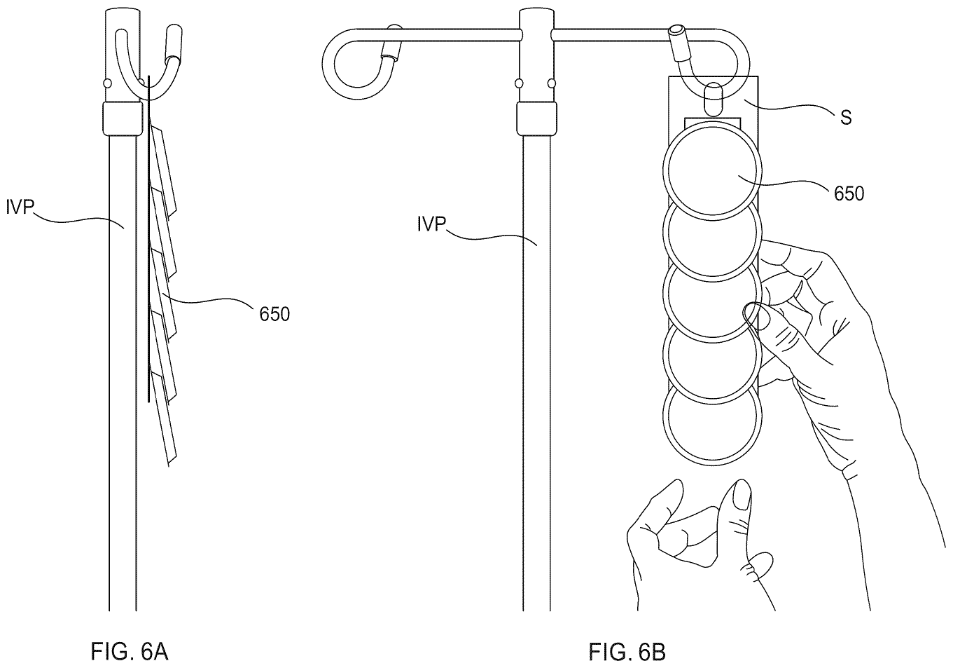

The encasement devices as described herein can be packed in individual packs. Together these packs can form a kit. For compact storage, the sleeves described herein can be collapsed and/or folded. FIG. 16 is an illustration of a sleeve 1610 in a collapsed or folded configuration, according to an embodiment. As shown, the sleeve 1610 is folded in a repeating concentric pushed-in pattern. In other words, a first portion of the sleeve 1610 can be folded into the inside of a second portion of the sleeve 1610, and the second portion of the sleeve can be folded into the inside of a third potion of the sleeve, and so on. Said another way, the sleeve 1610 is collapsed into itself via a series of Z-shaped folds. The collapsed configuration can be useful for storage or transport of the sleeve 1610. In some embodiments, the sleeve 1610 can more easily be placed into a package (e.g., packs 650, as described below with reference to FIGS. 6A and 6B) in its collapsed state. The collapsing can reduce the effective length of the sleeve 1610 during storage or transport by a factor of at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1,000.

FIGS. 6A and 6B are illustrations of a set of packs 650 for packaging of encasement devices, according to an embodiment. As shown, FIG. 6A is a side view of five packs 650 hanging from an IV pole IVP, while FIG. 6B is a front view of the packs 650 hanging from the IV pole IVP. Each pack 650 can include one encasement device, and the packs 650 can be arranged vertically on a single strip, with each of the packs 650 attached to a strip S. The strip S can be a tear away strip. In some embodiments, the packs 650 can be attached to the strip S via an adhesive. In some embodiments, the adhesive can be resealable. As shown, the strip S includes 5 packs 650. In some embodiments, the strip S can include 1, 2, 3, 4, 6, 7, 8, 9, 10, 11, 12, 13, 14, 15, 16, 17, 18, 19, 20, or more than 20 packs 650, inclusive of all values and ranges therebetween. In some embodiments, the packs 650 can include a tab disposed thereon for ease of opening. In some embodiments, the packs 650 can be composed of a material that can be torn open by hand. In some embodiments, multiple encasement devices can be disposed in each of the packs 650. In some embodiments, the packs 650 can include polybag materials (e.g., LDPE, LLDPE, MDPE, PP, PLA), foil, lined materials (i.e., materials having an outer layer and an inner lining), barrier pouches, cellulose packaging, or any other suitable materials or combinations thereof. In some embodiments, the packs 650 can include plastic encasements (e.g., hard plastic encasements), PP, HDPE, polybags inside a paperboard or cellulose and/or fiber-based container, or combinations thereof.

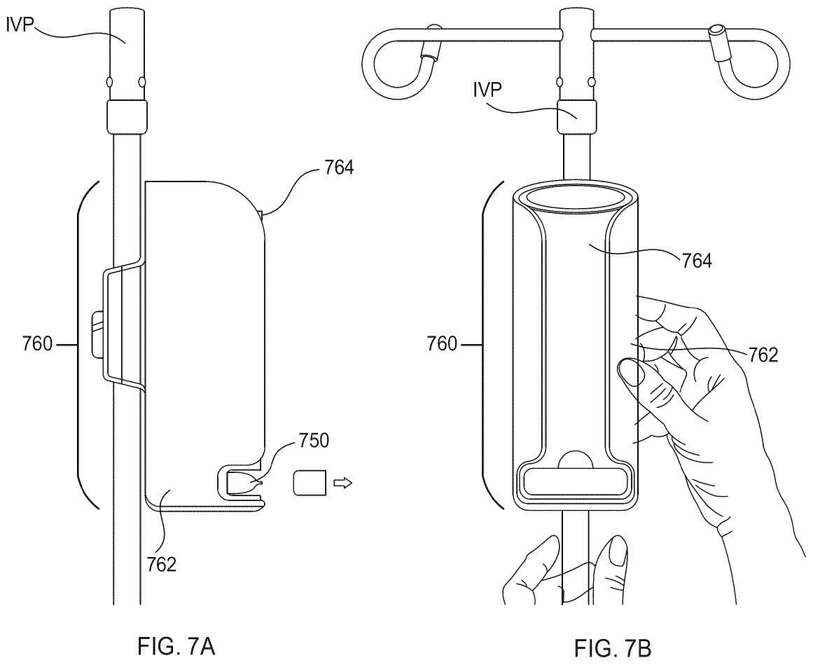

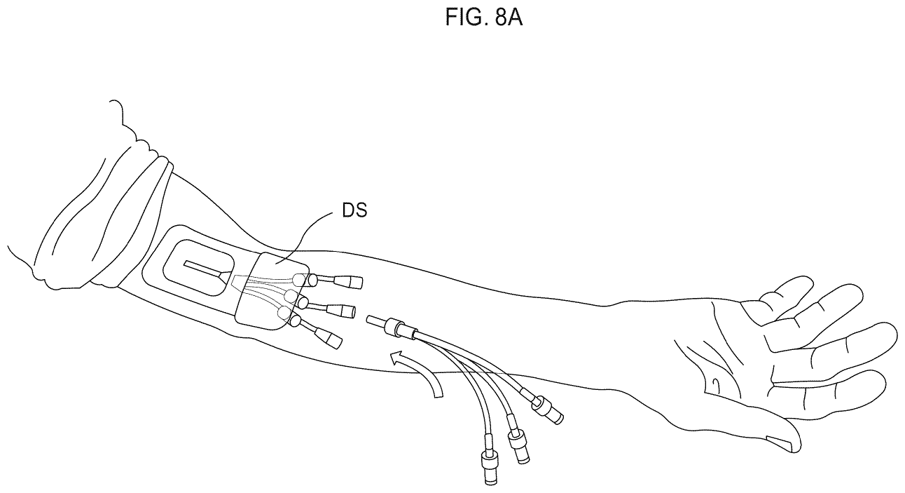

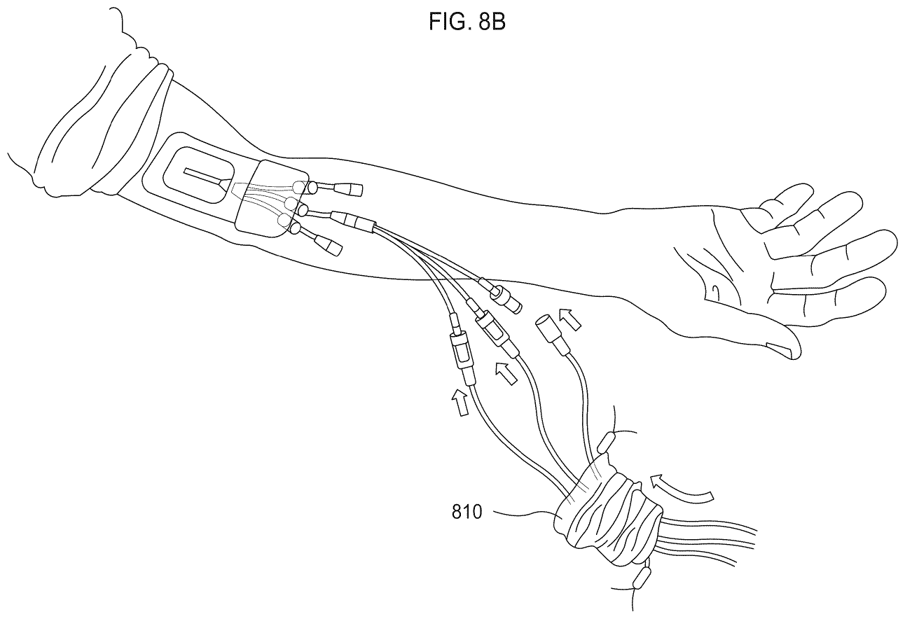

In some embodiments, a dispenser can be used to dispense one or more packs including encasement devices described herein. FIGS. 7A and 7B are illustrations of a system for dispensing of encasement packs 750, according to an embodiment. As shown, the packs 750 are disposed in a dispenser 760. The dispenser 760 includes a casing 762 and a box 764 and is suspended from an IV pole IVP. FIG. 7A is a side view of the system while FIG. 7B is a front view of the system. In some embodiments, the packs 750 can be the same or substantially similar to the packs 650, as described above with reference to FIGS. 6A-6B. Thus, certain aspects of the packs 750 are not described in greater detail herein.

As shown, the box 764 fits in the casing 762 and can be opened to dispense the packs 750. As shown, the packs 750 are arranged in a stacked configuration in the box 764. As shown, the box has a removable section that can be removed from the front to gain access to the packs 750. In some embodiments, the box 764 can be composed of cardboard, corrugated cardboard, or any other material that can be perforated, such that a section can be removed to access the packs 750. In some embodiments, the casing 762 can be composed of a polymer, a plastic, an injected molded plastic, or any other suitable material or combinations thereof. In some embodiments, the dispenser 760 can be sized to hold about 2, about 3, about 4, about 5, about 6, about 7, about 8, about 9, about 10, about 11, about 12, about 13, about 14, about 15, about 16, about 17, about 18, about 19, or about 20 packs 760, inclusive of all values and ranges therebetween. In some embodiments, the box 764 may be omitted or removed prior to the packs 750 being placed within the dispenser 760. As such, the individual packs 750 can be placed directly within the dispenser 760 without the presence of a box. While a box 764 is described, it can be appreciated that any other type of enclosure or housing can be used to contain one or more packs 750, including, for example, flexible containers such as bags, meshes, etc. In some embodiments, the box 764 can include a paperboard tube of individually wrapped sleeves (i.e., wrapped in the packs 750). In some embodiments, the box 764 can be replaced once all of the sleeves in the packs 750 are consumed.

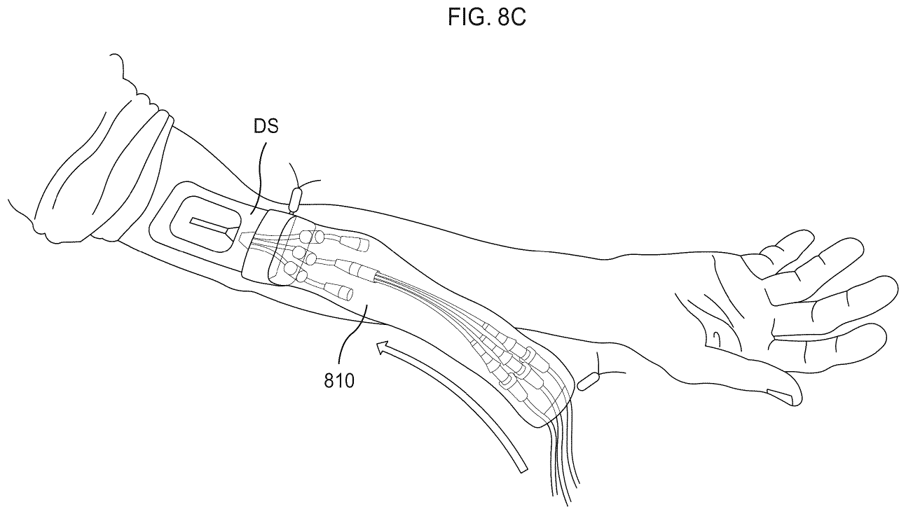

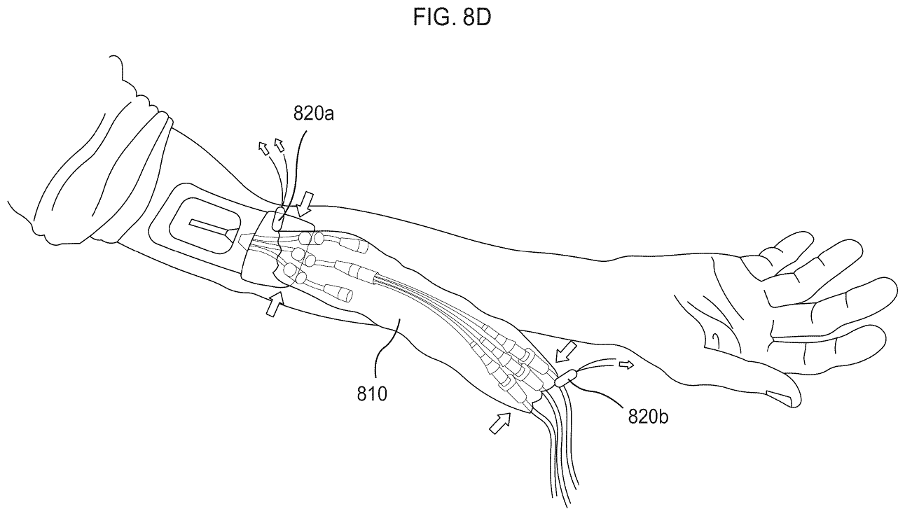

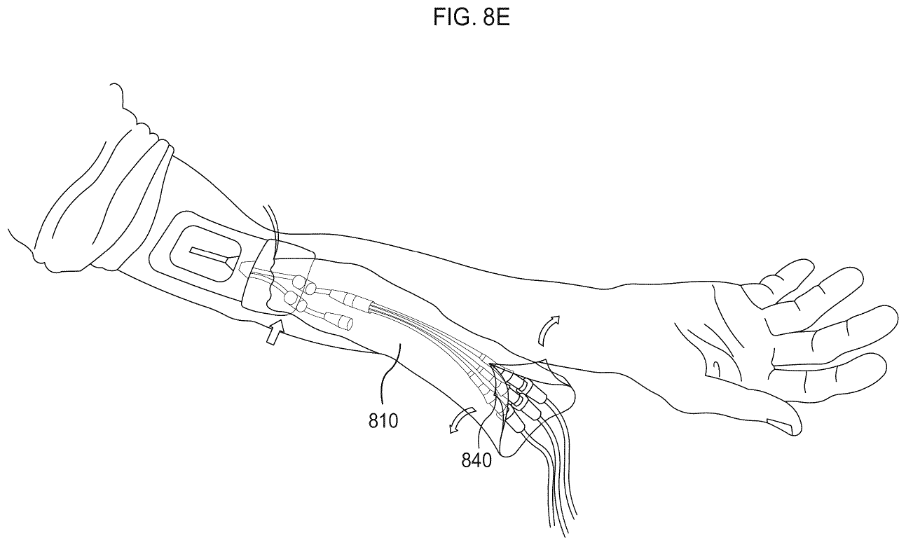

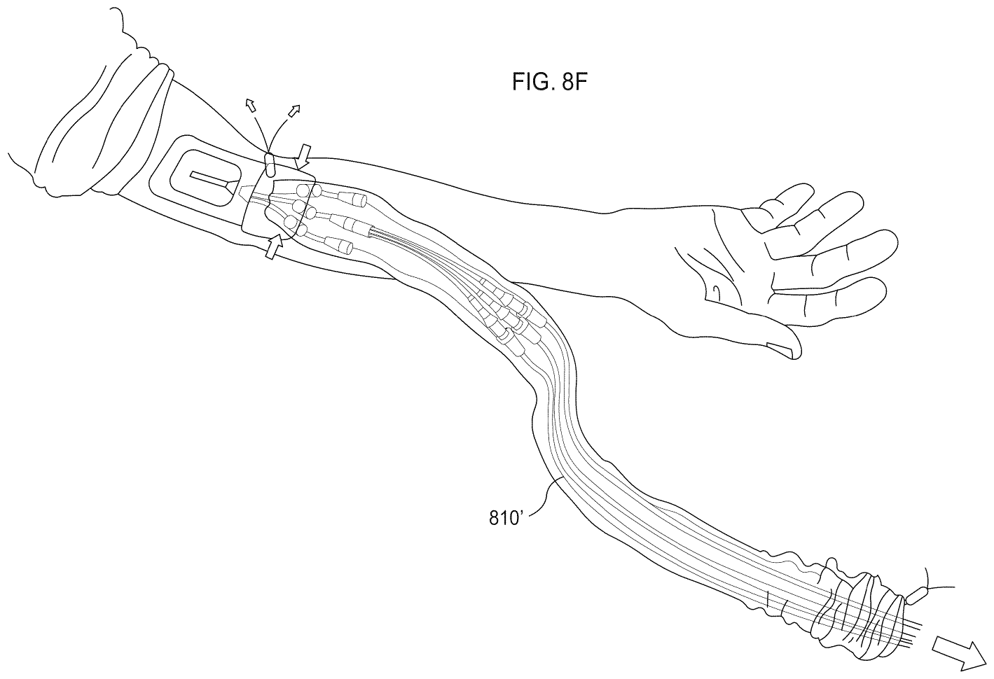

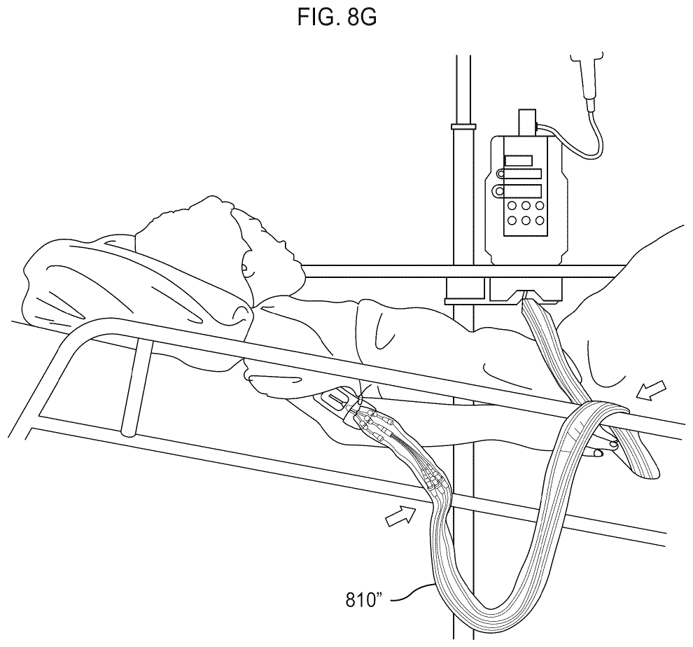

FIGS. 8A-8E are illustrations of a method of deploying an encasement device to protect IV lines from contaminants, according to various embodiments. As shown, the method includes attaching a central line connection to an IV line connection port in FIG. 8A. FIG. 8B shows the feeding of IV lines through a sleeve 810. The sleeve 810 can include components that are functionally and/or structurally similar to other sleeves described herein. FIG. 8C shows an extension of the sleeve 810 over IV line access ports and to a dressing DS. FIG. 8D shows securement of the sleeve 810 over a first region along the central lines near the dressing and over a second region along the IV lines and distal to the first region. The sleeve 810 can have two closing elements 820a, 820b, which can be configured to close around the central lines and/or IV line(s) at the first and second regions. In an embodiment, the closing elements can be implemented as snap-fit buttons. FIG. 8E shows removal of the sleeve 810 from the central lines and IV lines via tearing along perforations 840.

FIG. 8F shows extension of a long sleeve 810′ along the length of the IV lines and central lines. The sleeve 810′ can be a longer version of the sleeve 810 depicted with respect to FIGS. 8A-8E and can be deployed in a similar manner to sleeve 810. The sleeve 810′, however, has a longer length and therefore can cover lines having longer length. FIG. 8G shows a patient with a sleeve 810″ employed, with a proximal end of the sleeve 810″ cinched near the dressing and a distal end of the sleeve coupled closest to an IV pump. The sleeve 810′ can be a longer version of the sleeve 810 depicted with respect to FIGS. 8A-8E and can be deployed in a similar manner to sleeve 810. The sleeve 810′, however, has a length that can extend from the dressing to the IV pump.

While FIGS. 8A-8G are described with reference to a central line, it can be appreciated that the sleeves 810, 810′, 810″ can be used with other types of lines or tubing, including peripheral lines, IV lines, arterial lines, etc.

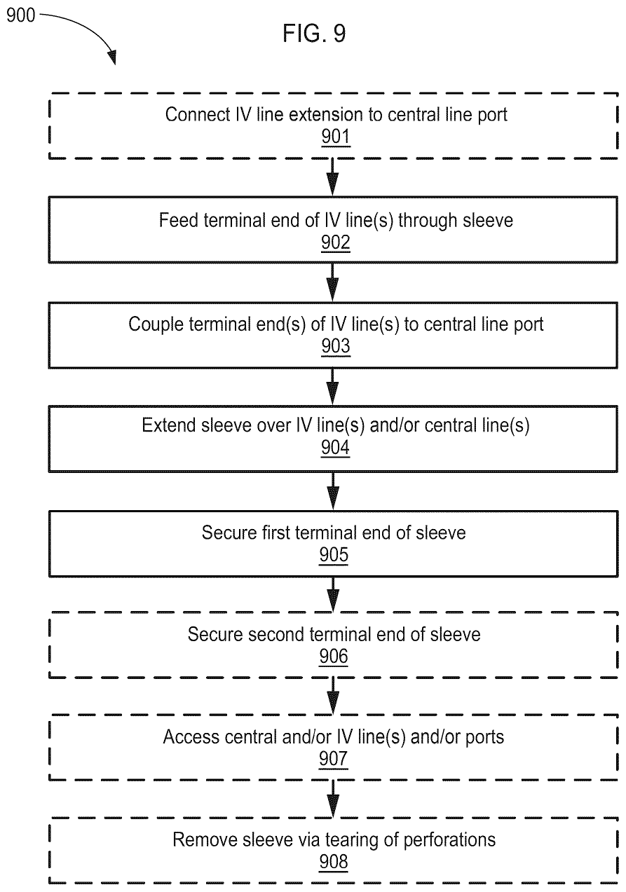

FIG. 9 is a block diagram of a method 900 of deploying an encasement device (e.g., such as any of the encasement devices or sleeves described herein) to protect a central line from contaminants, according to an embodiment. As shown, the method 900 optionally includes connecting an IV line extension to a central line port, at 901. The method 900 further includes feeding a terminal end of one or more IV lines through the sleeve, at 902, coupling the terminal ends of the IV line to the central line port, at 903, extending the sleeve over the central lines and central line ports and over at least a portion of the IV lines, at 904, and securing the first terminal end of the sleeve, at 905. The method 900 optionally includes securing a second terminal end of the sleeve, at 906, accessing the central line and/or IV line(s) and/or ports, at 907, and removing the sleeve, at 908.

At 901, an IV line extension is optionally connected to a central line port. The central line port can come from a dressing, and the IV extension piece can allow for multiple IV lines to be fed to a single central line port in the patient's body. At 902, a terminal end of the IV line is fed through a sleeve (e.g., the sleeve 110, as described above with reference to FIG. 1). In some embodiments, the sleeve can be compressed or bunched together as the central line(s) is/are fed through the sleeve. In some embodiments, the sleeve can be fully extended as the IV line(s) is/are fed through the sleeve.

The terminal end of the IV line is coupled to the central line port, at 903. In some embodiments, if the IV extension piece was connected to the central line port at 901, 903 can include coupling the central lines to the IV extension piece. The coupling at 903 can establish a fluidic pathway from a source (e.g., an IV bag, pump, or other device) to a patient's body. At 904, the sleeve extends over at least a portion of the central line(s) that is not covered by the central line dressing, and extends over at least a portion of the IV line. This extension protects multiple points along the IV line(s) that would otherwise be susceptible to contamination. In some embodiments, the extension can extend from the dressing to an IV pump. In some embodiments, the extension at the proximal end can go to the dressing. In some embodiments, the extension at the proximal end can go to a location near the dressing, e.g., a location distal to the dressing by a distance of about 1 cm, about 2 cm, about 3 cm, about 4 cm, about 5 cm, about 6 cm, about 7 cm, about 8 cm, about 9 cm, or about 10 cm, inclusive of all values and ranges therebetween. In some embodiments, the distal end of the sleeve can extend to a location distal to each connection point along the IV lines. In some embodiments, the distal end of the sleeve can extend to the IV pump. In some embodiments, the distal end of the sleeve can extend to a location proximal to the IV pump by about 1 cm, about 2 cm, about 3 cm, about 4 cm, about 5 cm, about 6 cm, about 7 cm, about 8 cm, about 9 cm, about 10 cm, about 20 cm, about 30 cm, about 40 cm, about 50 cm, about 60 cm, about 70 cm, about 80 cm, about 90 cm, about 1 m, about 2 m, about 3 m, about 4 m, about 5 m, about 6 m, about 7 m, about 8 m, about 9 m, or about 10 m, inclusive of all values and ranges therebetween.

A first terminal end of the sleeve is secured, at 905. In some embodiments, the first terminal end of the sleeve can be the proximal end of the sleeve. In some embodiments, the first terminal end of the sleeve can be secured to the dressing. In some embodiments, the first terminal end of the sleeve can be secured to a location near the dressing (e.g., around the outside of the IV lines at a location distal to but near the dressing). In some embodiments, the coupling of the first terminal end can be via an adhesive, a drawstring, a cinch, an adhesive flange, and/or any of the other mechanisms described above with reference to the closing elements 120, described with reference to FIG. 1.

The second terminal end of the sleeve is optionally secured, at 906. In some embodiments, the second terminal end of the sleeve can be the distal end of the sleeve. In some embodiments, the second terminal end of the sleeve can be secured to an IV pump. In some embodiments, the second terminal end of the sleeve can be secured to a location proximal to the IV pump (e.g., around the outside of the IV at a location proximal to but near the IV pump), e.g., by about 1 cm, about 2 cm, about 3 cm, about 4 cm, about 5 cm, about 6 cm, about 7 cm, about 8 cm, about 9 cm, about 10 cm, about 20 cm, about 30 cm, about 40 cm, about 50 cm, about 60 cm, about 70 cm, about 80 cm, about 90 cm, about 1 m, about 2 m, about 3 m, about 4 m, about 5 m, about 6 m, about 7 m, about 8 m, about 9 m, or about 10 m, inclusive of all values and ranges therebetween. In some embodiments, securing the second terminal end of the sleeve can be via an adhesive, a drawstring, a cinch, an adhesive flange, and/or any of the other mechanisms described above with reference to the closing elements 120, described with reference to FIG. 1. In some embodiments, the second terminal end of the sleeve can be a closed end and therefore the second terminal end of the sleeve may not need to be secured.

The method 900 optionally includes accessing the central line and/or IV line and/or ports, at 907. In some embodiments, the central line can be accessed while the sleeve is covering the central line and IV line(s). For example, the central line and IV line(s) can be accessed through a flap in a sidewall of the sleeve. In some embodiments, the central line and IV line(s) can be accessed by opening one or more of the closing features (e.g., cinch). Opening one or more of the closing features can allow a medical professional to roll the sleeve out of the way to access the central line and IV line(s), and then roll the sleeve back into place to re-cover the central line and IV line(s).

The sleeve is optionally removed, at 908. In some embodiments, removal of the sleeve can be via tearing of one or more perforations along the length and/or perimeter of the sleeve. In some embodiments, the removal of the sleeve can be via unthreading the IV lines and pulling them through the sleeve and out of the sleeve.

While FIG. 9 is described with reference to a central line and IV line(s), it can be appreciated that method 900 can involve other types of lines or tubing, including peripheral lines, arterial lines, hemodialysis lines, etc.

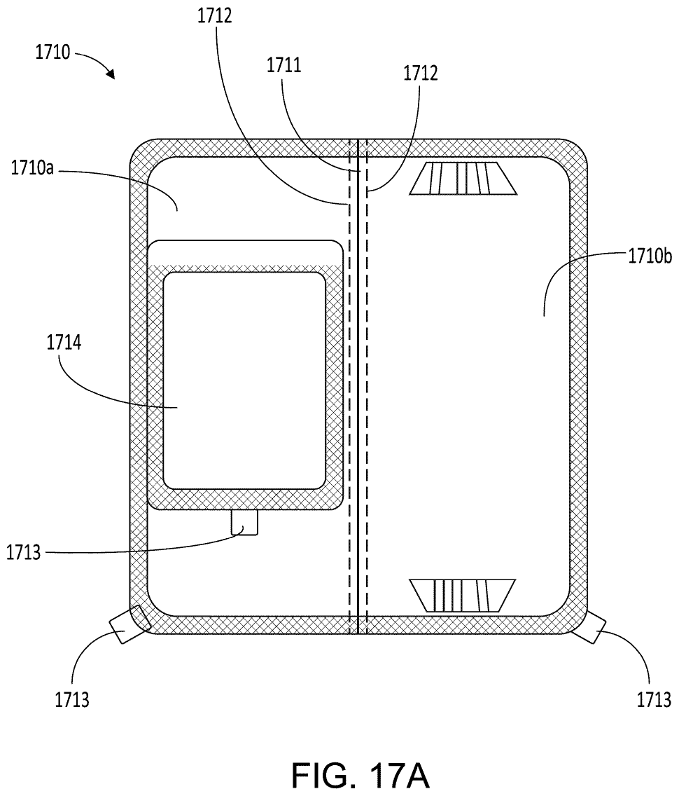

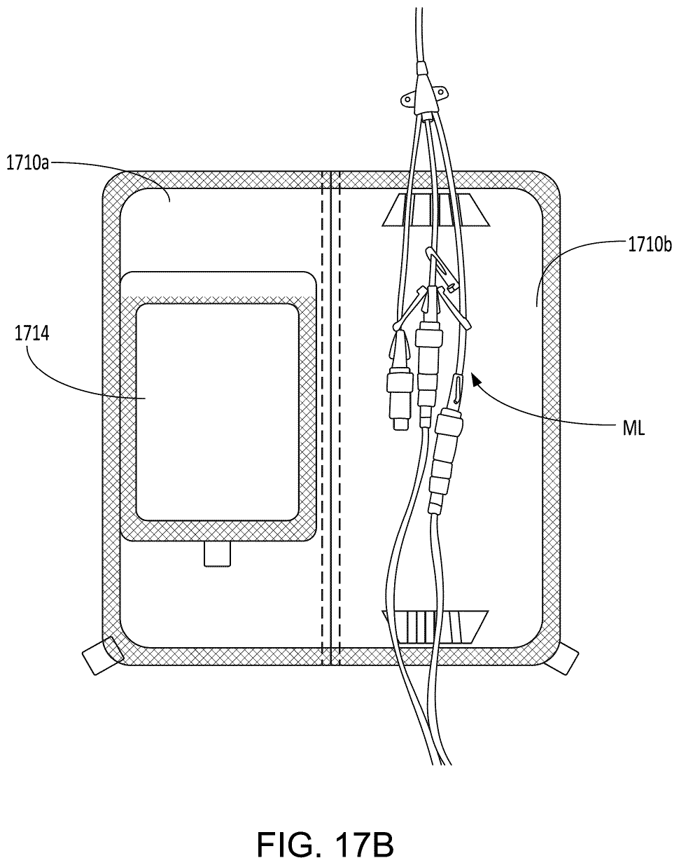

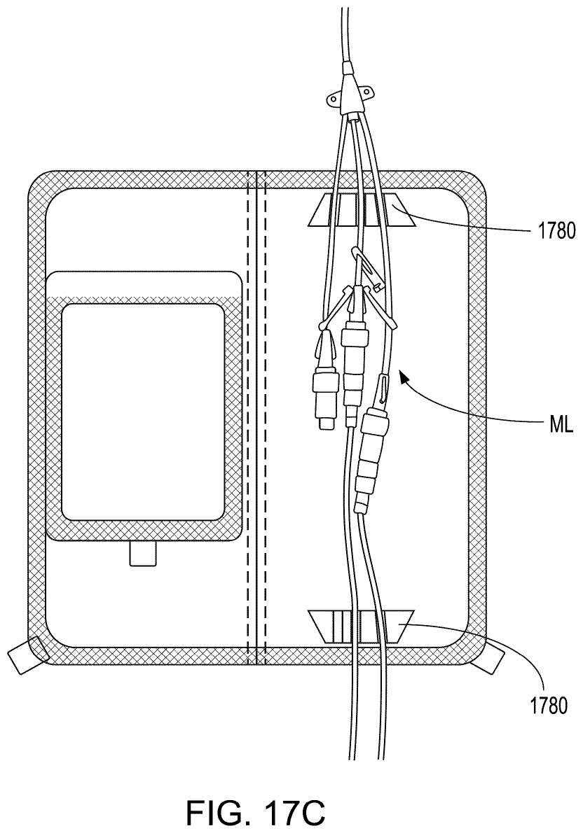

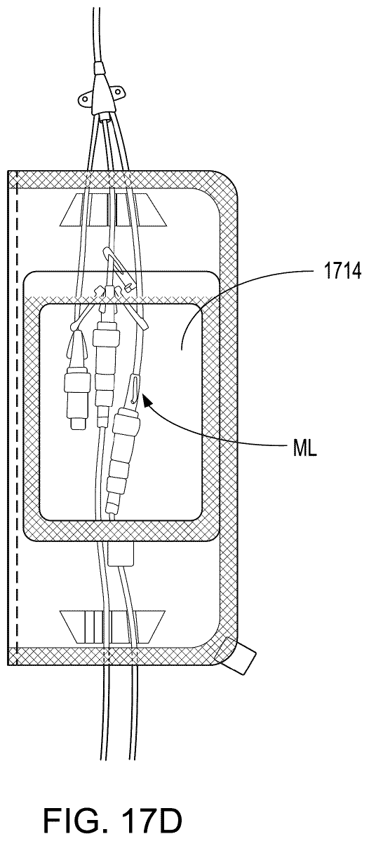

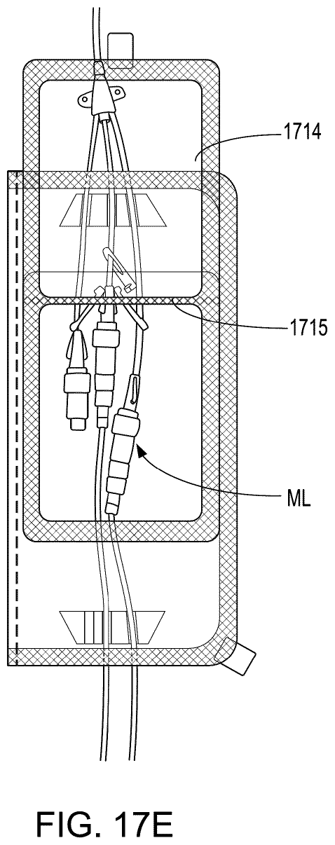

FIGS. 17A-17E are illustrations of an encasement or sleeve 1710, according to an embodiment. As shown, the encasement 1710 includes a first section or part 1710a and a second section or part 1710b that are coupled together. The encasement 1710 can be functionally and/or structurally similar to other encasements or sleeves described herein, including, for example, encasement 110. Thus, certain aspects of the encasement 1710 are not described in greater detail herein.

The first and second sections 1710a, 1710b can fold over a set of one or more medical lines (ML). The encasement 1710, by being designed as two sections that can be folded over to create an enclosure around a set of lines, can provide certain advantages. For example, the encasement 1710 can be manufactured as one or more sheets or flat sections that can be coupled together or folded along a seam. In some embodiments, the coupling or fold can also provide greater strength or rigidity to the sleeve, which can, for example, facilitate application and/or usage of the encasement 1710. The flat nature of the encasement 1710 can also reduce the profile of the encasement 1710, e.g., enabling easier storage.

The first section 1710a (e.g., a top part) can be configured to fold over the second section 1710b (e.g., a bottom part). The first section 1710a can be made of a see-through material (e.g., a transparent or translucent material) to allow for visibility of the set of lines ML, which are positioned between the first and second sections 1710a, 1710b. In some embodiments, the first section 1710a can include a window with a flap or cover 1714 that can be made of a transparent material, e.g., to allow for visibility of the set of lines and any ports underneath the first section 1710a when the first section 1710a has been folded over the second section 1710b, as shown in FIG. 17D. In some embodiments, the flap 1714 can be reversibly opened and closed. In some embodiments, the flap 1714 can be coupled to the first section 1710a via an adhesive, or otherwise fastened, bonded, or attached to the first section 1710a. The flap 1714 can be opened to provide unobstructed access to the lines ML and ports, without having to remove the entire encasement 1710 or having to unfold or remove the first section 1710a.

As shown in FIGS. 17A-17E, the window or flap 1714 can be positioned over an opening that is approximately in the middle part of the first section 1710a. Alternatively, the window can be positioned higher or lower, e.g., depending on the type of line(s) that the encasement 1710 may be used to protect and/or other factors (e.g., where the encasement 1710 may be placed relative to the lines ML and ports, the length of the encasement 1710, etc.). For example, with a shorter hemodialysis line(s) and/or catheter, the window can be positioned higher (e.g., toward a patient heard and/or line insertion point). Alternatively, with a central line(s), PICC lines and/or ports, the window can be approximately in the middle or lower. The window can be configured to open in one or more ways. For example, flap 1714 may be coupled to the first section 1710a via a hinge 1715 that can be positioned to the left, the right, the top, or the bottom of the flap 1714. As depicted in FIG. 17E, the hinge 1715 can be positioned to the top of the flap 1714. The flap 1714 can have resealable adhesive along its three free sides (i.e., the sides that do not form the hinge 1715) that allow the flap 1714 to reversibly close and open. The flap 1714, by being resealable, can allow for access and/or handling of uncontaminated lines ML and ports, thereby decreasing the risk of bio-burden and pathogen transference when handling the lines ML and/or ports. For example, the flap 1714 can be opened to allow for access to a port for infusion, and then when the infusion is finished, the flap 1714 can be closed. As such, the encasement 1710 can be suited for patients that require intermittent infusions.

The second section 1710b, or bottom part of the sleeve, can include a section that includes color, e.g., to provide visual contrast to the lines ML placed above it. This bottom or base layer may be colored by pigment during extrusion and/or a coating or label applied during device fabrication or preparation. In some embodiments, the second section 1710b can be implemented as a thin film or other flexible material. In some embodiments, the second section 1710b can also include an adhesive for anchoring the encasement 1710 to a patient or other surface. This adhesive can be disposed on an outer facing surface (i.e., surface that faces away from the lines ML) of the second section 1710b.

The seam or fold 1711 can provide the encasement 1710 with addition strength or stiffness, e.g., to maintain a shape of the encasement 1710. The seam 1711 can provide a pre-determined hinging motion between the first and second sections 1710a, 1710b, thereby allowing for sealing alignment between the first and second sections 1710a, 1710b when applying the encasement 1710 around a set of lines ML. In particular, the seam 1711 can match the top part or exposed part of the adhesive or other closure mechanism of the first and section sections 1710a, 1710b when the first section 1710a is folded over the second section 1710b.

In some embodiments, there can be one or more perforations 1712 on the first and/or second sections 1710a, 1710b. The perforation(s) 1712 can be, for example, next to the seam 1711 on the first and/or second sections 1710a, 1710b. The perforation(s) 1712 can allow for simple and/or fast removal of the encasement 1710 when the encasement 1710 is no longer needed. In some embodiments, additional perforations can be provided as secondary and/or backups for quick removal of the encasement 1710.

In some embodiments, the encasement 1710 can include one or more tabs 1713. The tab(s) 1713 can be located on one or both sections 1710a, 1710b. In some embodiments, there can be tabs 1713 located on a bottom side of each section 1710a, 1710b. In some embodiments, there can be tabs 1713 located on a top side of each section 1710a, 1710b. In some embodiments, multiple tabs 1713 can be located on the top, bottom, middle, and/or other sides of the first and second sections 1710a, 1710b. In particular, the location of the tabs 1713 can be anywhere along the open side of the encasement 1710 in order to help with easy opening of the encasement 1710. These tabs 1713 have no adhesive and can facilitate a user with pulling apart the first and second sections 1710a, 1710b. In some embodiments, a tab 1713 can also be located on the flap 1714, e.g., to pull open the window.

To apply the encasement 1710, the encasement can be laid open, e.g., as depicted in FIG. 17A. The second section 1710b (or bottom section) can be placed under a set of lines ML, as depicted in FIG. 17B. The lines ML can be organized into one or more holders 1780, as depicted in FIG. 17C. In some embodiments, the holder 1780 can be the same or substantially similar to the holder 1580, as described above with reference to FIGS. 15A-15B. The holder 1780 can be coupled to an interior surface of the encasement 1710. In some embodiments, the holder 1780 can be coupled to the interior surface of the encasement 1710 via an adhesive or other fastening component. The folding design of the encasement 1710 can allow for easier installation of the holder 1780 during manufacture. For example, the holder 1780 can be placed on and coupled to an interior surface of the encasement 1710 when the two sections 1710a, 1710b of the encasement 1710 are laid open. The first section 1710a can then be folded over the second section 1710b, aligned (e.g., with the bottom edges), and the first and second sections 1710a, 1710b can be sealed together, e.g., with a resealable adhesive. The adhesive can be resealable, similar in strength to resealable adhesive used in sanitary wipe packaging (e.g., an adhesive pad) or other similar sealing mechanism (e.g., Ziploc, or other mating seals). This folded configuration is depicted in FIG. 17D. The encasement 1710, once folded over or closed, can minimize line and/or port exposure to patient and environmental hazards and bio-burden pathogens, thereby decreasing the risk of biofilm buildup or pathogen transference when handling lines and injecting into ports.

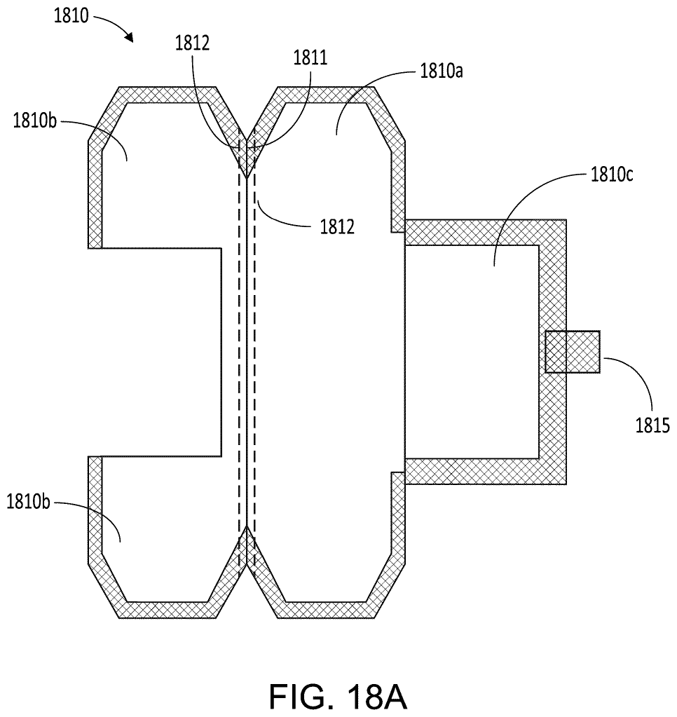

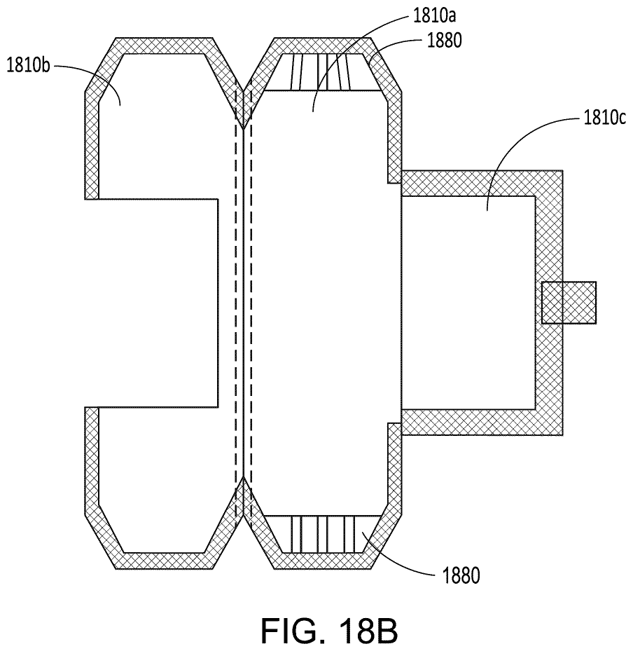

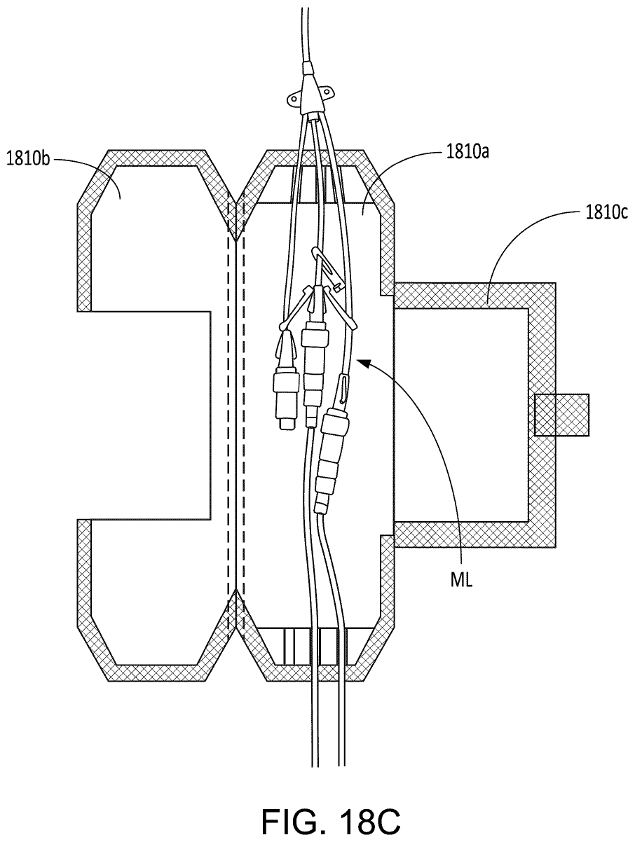

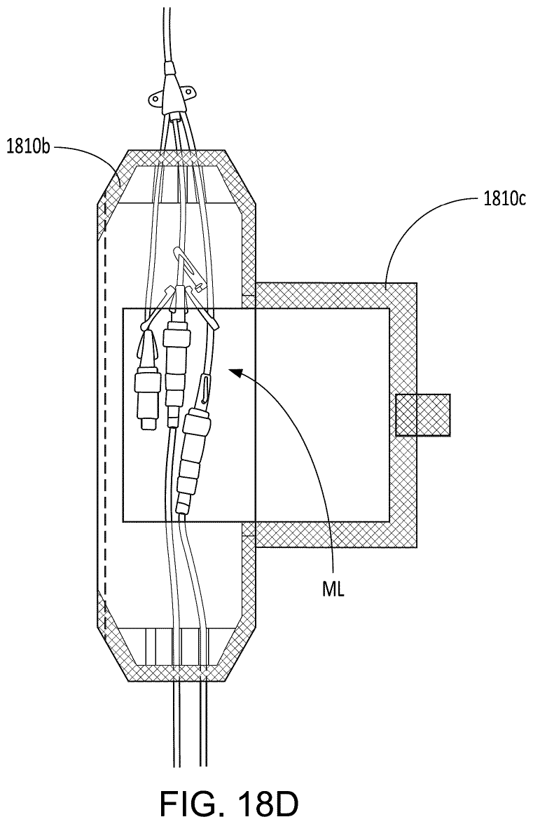

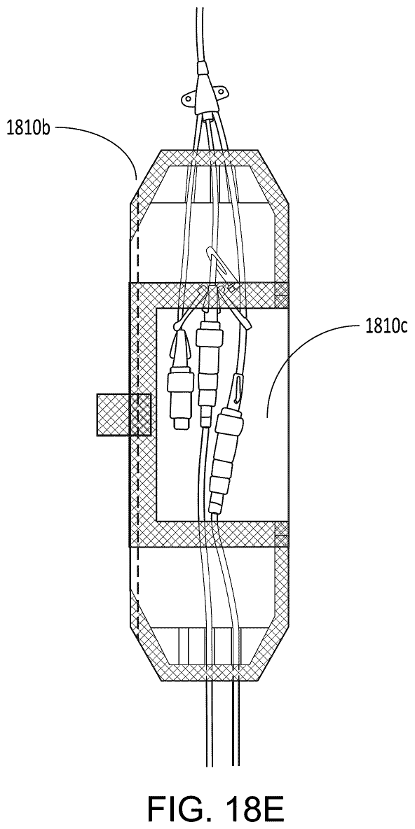

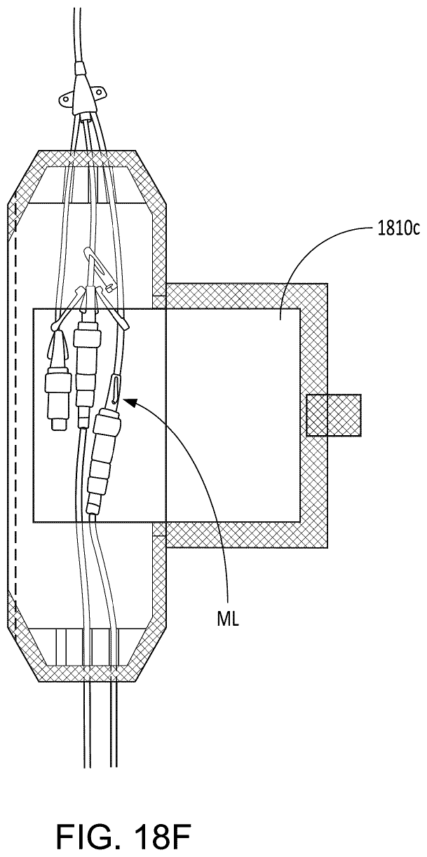

FIGS. 18A-18F are illustrations of an encasement or sleeve 1810, according to an embodiment. The encasement 1810 can be structurally and/or functionally similar to other encasements or sleeves described herein, including, for example, encasement 110 and/or 1710. Thus, certain aspects of the encasement 1810 are not described in greater detail herein. The encasement 1810, however, can include three parts with one or more folding parts.

Similar to the sleeve 1710, the encasement 1810 can include a section 1810a that is designed to go under a set of medical lines ML. This section 1810a can include color, e.g., to provide visual contrast to the lines ML placed over it.

The encasement 1810 can also include a section or layer 1810b that is configured to fold over the section 1810a. The section 1810b can be similar to the section 1710a (described above with reference to FIGS. 17A-17E) but include two parts, e.g., a top part and a bottom part, which are separated by an opening. The section 1810b can be configured to fold along a seam 1811 and seal along the edges of section 1810a. In use, the top and bottom parts of section 1810b can be individually or separately opened and resealed. This can allow access to the medical lines ML individually and/or to add additional lines. For example, to add another line to the ports, a user can open the bottom part of the section 1810b while keeping the top part closed, e.g., to ensure continuous antimicrobial protection of the lines and/or ports that are not needed for a particular procedure. Similar to the section 1710a described with reference to FIGS. 17A-17E, the section 1810b can be made of a transparent or translucent material, e.g., to have visibility of the lines ML and/or ports when the section 1810b has been folded over.

The encasement 1810 can include a third section 1810c, which can function as a resealable window. Similar to the window described with reference to FIGS. 17A-17E, the window 1810c can be made of a transparent or translucent material, e.g., to have visibility of the lines ML and/or ports when the window is closed. The resealable window 1810c allows for access/handling of uncontaminated lines and/or ports, thereby decreasing the risk of bio-burden pathogen transference when lines are handled for injecting into ports.

The encasement 1810 can include an adhesive, which can be placed along a perimeter of the encasement 1810, and/or other overlapping areas, e.g., to ensure the edges and covering encasing and protecting the lines ML form a continuous effective barrier around the outer edges of the encasement 1810. The adhesive can be resealable, e.g., similar in strength to the resealable adhesive used in wet wipes or other sanitary wipes packaging. The encasement 1810 can also include perforation(s) 1812 and/or tab(s) 1815, similar to that described with reference to the encasement 1710 in FIGS. 17A-17E.

In some embodiments, the encasement 1810 can include one or more holders 1880, as depicted in FIG. 18B. The holders 1880 can be on the upper and lower end of the section 1810a. The holders 1880 can ensure alignment of the lines ML and/or ports as well as the protective seal. The holders 1880 can be different sizes and/or include different slot sizes, e.g., to accommodate different size and/or type of lines. The material of the holders 1880 can be non-absorbent foam, silicone, anti-microbial material, or the like, e.g., to prevent biofilm migration transfer from the line outside the encasement 1880. The line holders 1880 may come pre-plugged, or precut, with plug material that can be removed during device application, e.g., allowing the medical care professional to determine if 1, 2, 3, etc. lines are needed.

To apply the encasement 1810, the encasement can be laid open, e.g., as depicted in FIG. 18A. The lines ML can be placed on top of section 1810a, as depicted in FIG. 18C. The section 1810b can be folded over and attached to section 1810a, as depicted in FIG. 18D. The section 1810c can be folded over and attached to section 1810a and/or section 1810b, as depicted in FIG. 18E. In use, section 1810b can be folded inward over section 1810a, and subsequently, section 1810c can be folded over section 1810b, e.g., to create the window. The sections 1810a, 1810c can then be selectively opened, e.g., to provide access to lines and/or ports underneath.

While encasement 1710 is depicted as having two parts and encasement 1810 is depicted as having three parts, it can be appreciated that an encasement as described herein can include any number of parts. In some embodiments, an encasement can have more than three parts and/or sections, which can each individually open, e.g., to provide access to lines and/or ports encased or sealed within different regions of the encasement. While not depicted in FIGS. 17A-18F, it can be appreciated that encasements 1710 and 1810 can include other components, e.g., closing elements, flanges, etc.

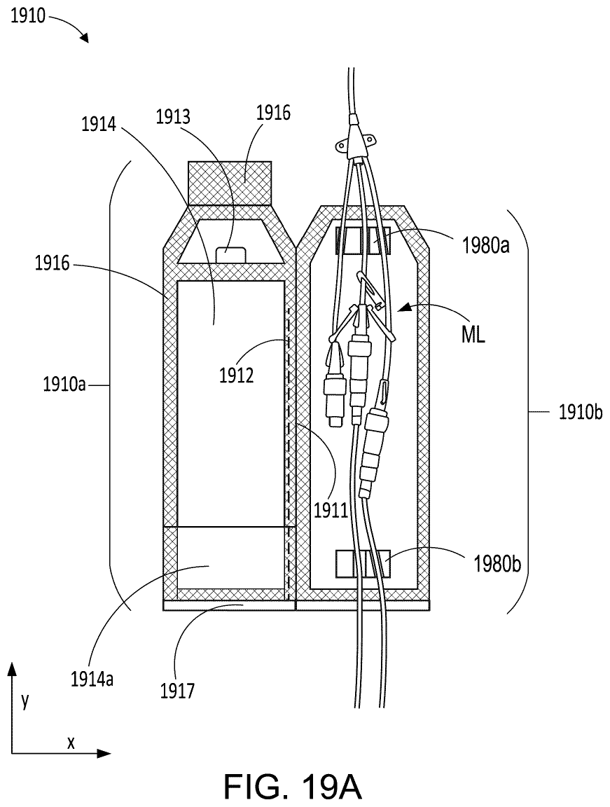

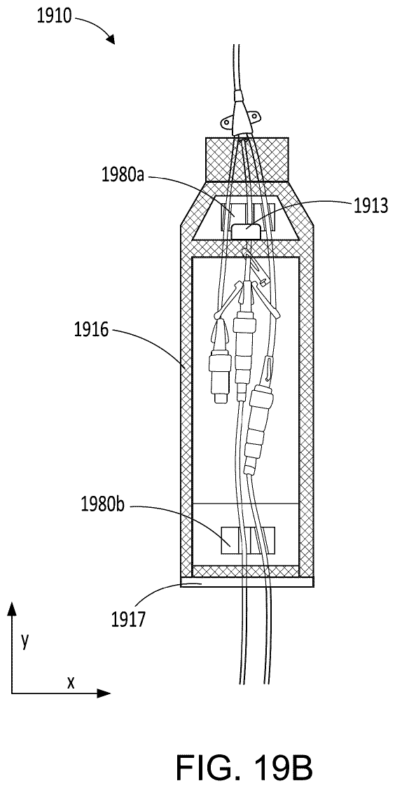

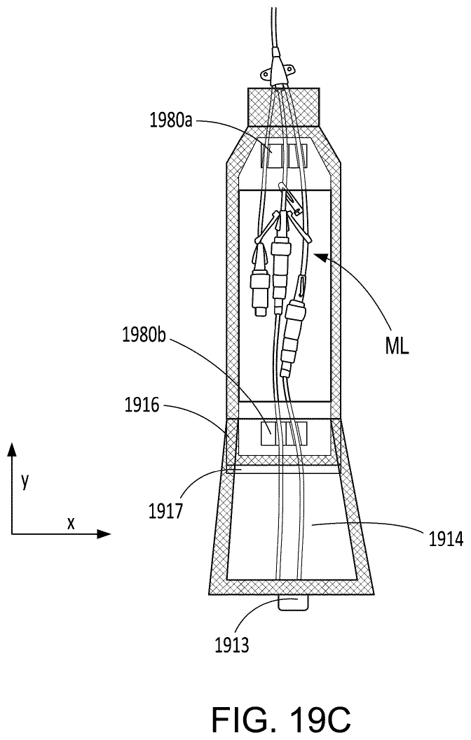

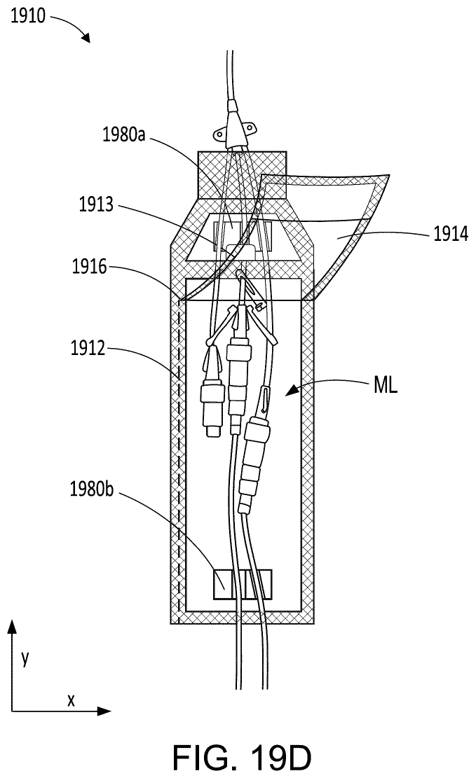

FIGS. 19A-19D are illustrations of an encasement 1910 implemented as a foldable sleeve, according to an embodiment. As shown, the encasement 1910 includes a section 1910a and a section 1910b. The encasement 1910 can be structurally and/or functionally similar to other encasements or sleeves described herein, including, for example, encasement 110 and/or 1710. FIG. 19A shows the section 1910a and the section 1910b lying flat, in an open configuration. FIG. 19B shows the section 1910a folded over onto the section 1910b. FIG. 19C shows a flap 1914 of the encasement 1910 in an open configuration. FIG. 19D shows the section 1910a separated from the section 1910b. Axes are shown for visual clarity.

The section 1910a and the section 1910b are joined along a seam 1911. A perforation 1912 extends at least partially along a length of section 1910a. Alternatively, in some embodiments, a perforation can extend at least partially along a length of the section 1910b and/or both sections 1910a, 1910b. In some embodiments, the section 1910a can fold over onto the section 1910b. As shown in FIG. 19C, the section 1910a includes a flap or cover 1914 that can be opened to allow access to medical lines ML that are disposed between sections 1910a, 1910b. The flap 1914 can be attached and detached from the section 1910a via movement of a tab 1913. The section 1910a is coupled (e.g., adhered, bonded) to the section 1910b via an adhesive surface 1916. In some embodiments, a non-adhesive region 1917 is included on the surface of the section 1910a and the section 1910b, toward one side of the sections (e.g., a bottom of the sections, as shown in FIGS. 19A-19D), such that the non-adhesive regions 1917 can function as tabs that can be used to pull the two sections 1910a, 1910b apart, after they have been coupled to one another. In some embodiments, the encasement 1910 can include a top or proximal holder 1980a and a bottom or distal holder 1980b (collectively referred to as holders 1980). In some embodiments, the encasement 1910 can include a single holder (e.g., a top or a bottom holder), or additional holders 1980.

In some embodiments, the section 1910b can be tinted or colored, e.g., to provide visual contrast to the medical lines ML. In some embodiments, the section 1910b can include a film or adhesive label that has an amount of color or pigmented to provide visual contrast. In some embodiments, the section 1910b can be coated with color to provide visual contrast. In some embodiments, the section 1910b can include a label. In some embodiments, the coating or pigment can be applied during extrusion. In some embodiments, the label can be applied during device preparation or fabrication. In some embodiments, the underside of the section 1910b can include an adhesive to anchor the encasement to the patient, e.g., for functionality and/or usability purposes.

As shown, the seam 1911 extends along a side of the section 1910a and the section 1910b. As shown, the seam 1911 extends along a majority of the length of the section 1910a and the section 1910a, joining that portion together, while a small section of the length of the section 1910a and the section 1910b are not joined. The seam 1911 has a length, along which the section 1910a is coupled to the section 1910b. In other words, the section 1910a and the section 1910b have an adjoining length. In some embodiments, the seam 1911 can give the encasement 1910 additional stiffness to maintain the shape of the encasement 1910, e.g., to prevent undesirable wrinkling or folding of the encasement 1910 along its length. In some embodiments, the seam 1911 can provide a pre-determined hinging motion and facilitate sealing alignment when applying the section 1910b to the section 1910a. This can aid in properly aligning the adhesive surface 1916 on the section 1910a and/or the section 1910b with the desired adherence location on the other side.

As shown, the perforation 1912 extends along the y-axis along the section 1910a and is offset from the seam 1911. In some embodiments, the perforation 1912 can extend or be disposed along the seam 1911. In some embodiments, the perforation 1912 can extend along the section 1910b at a location offset from the seam 1911. In some embodiments, the perforation 1912 can be offset from the seam 1911 by an offset distance of about 1 mm, about 2 mm about 3 mm, about 4mm, about 5 mm, about 6 mm, about 7 mm, about 8 mm, about 9 mm, about 1 cm, about 1.5 cm, about 2 cm, about 2.5 cm, about 3 cm, about 3.5 cm, about 4 cm, about 4.5 cm, or about 5 cm, inclusive of all values and ranges therebetween. As shown, the perforation 1912 extends along about 75% of the adjoining length between the section 1910a and the section 1910b. In some embodiments, the perforation 1912 can extend along the adjoining length between the section 1910a and 1910b by about 50% to about 100% of the adjoining length, including all sub-ranges and values therebetween. In some embodiments, the perforation 1912 may extend less than a full length of the adjoining length between the sections 1910a and 1910b such that after opening the sleeve along the perforation, the two section 1910a and 1910b can still be brought back together and coupled to one another, e.g., via adhesive. In other words, the perforation 1912 can be torn with the adhesive surface 1916 still intact without any partial or full replacement of the encasement 1910, and then the torn portion of the section 1910a (and/or the section 1910b) can be placed back in its original location via the adhesive surface 1916. In some embodiments, the perforation 1912 can allow for easier access of the medical lines ML. In some embodiments, the perforation 1912 can enable easier adding or removal of a line. Because the perforation runs from the bottom of the sleeve, it avoids having to insert a line under a portion of the sleeve when the line is being added. FIG. 19D depicts the sleeve with the section 1910a opened, after being torn along the perforation 1912.

In some embodiments, the perforation 1912 can extend along at least about 50%, at least about 55%, at least about 60%, at least about 65%, at least about 70%, at least about 75%, at least about 80%, or at least about 85% of the adjoining length between the section 1910a and the section 1910b. In some embodiments, the perforation 1912 can extend along no more than about 90%, no more than about 85%, no more than about 80%, no more than about 75%, no more than about 70%, no more than about 65%, no more than about 60%, or no more than about 55% of the adjoining length between the section 1910a and the section 1910b. Combinations of the above-referenced percentages are also possible (e.g., at least about 50% and no more than about 90% or at least about 60% and no more than about 80%), inclusive of all values and ranges therebetween. In some embodiments, the perforation 1912 can extend along about 50%, about 55%, about 60%, about 65%, about 70%, about 75%, about 80%, about 85%, or about 90% of the adjoining length between the section 1910a and the section 1910b.

In some embodiments, the perforation 1912 can extend from a first edge or side of the section 1910a and/or the section 1910b to a point separated from a second, opposite edge of the section 1910a and/or the section 1910b by a separation distance. In some embodiments, the separation distance can be at least about 1 cm, at least about 2 cm, at least about 3 cm, at least about 4 cm, at least about 5 cm, at least about 6 cm, at least about 7 cm, at least about 8 cm, or at least about 9 cm. In some embodiments, the separation distance can be no more than about 10 cm, no more than about 9 cm, no more than about 8 cm, no more than about 7 cm, no more than about 6 cm, no more than about 5 cm, no more than about 4 cm, no more than about 3 cm, or no more than about 2 cm. Combinations of the above-referenced separation distances are also possible (e.g., at least about 1 cm and no more than about 10 cm or at least about 2 cm and no more than about 6 cm), inclusive of all values and ranges therebetween. In some embodiments, the separation distance can be about 1 cm, about 2 cm, about 3 cm, about 4 cm, about 5 cm, about 6 cm, about 7 cm, about 8 cm, about 9 cm, or about 10 cm. In some embodiments, the separation distance can be small, e.g., less than about 5 cm, such that the section 1910a and the section 1910b can be separated from one another, and the entire sleeve be removed and disposed of.

As shown in FIGS. 19A-19C, the flap 1914 extends along the full width of the section 1910a. In some embodiments, the edges of the flap 1914 can be flush or approximately flush with the edges of the section 1910a and/or 1910b. The flap 1914 opens downward to expose the medical lines ML. In some embodiments, the flap 1914 can be transparent and act as a window for viewing of the medical lines ML. The transparency and openability of the flap 1914 can allow undisturbed vision of the medical lines ML and/or undisturbed access to the medical lines ML, without having to remove the entire encasement 1910 or having to tear open the perforation. In some embodiments, the flap 1914 can include a non-transparent or less transparent section 1914a, e.g., for use as a label, to identify a side (e.g., bottom side) of the flap, etc. The tab 1913 allows easy application and opening of the flap 1914. The tab 1913 creates a grip point for a medical professional when opening the flap 1910, e.g., to gain access to the medical lines ML and/or to administer medicine. The tab 1913 can be non-adhesive. In some embodiments, the flap 1914 can include multiple tabs 1913.

As shown, the flap 1914 overlaps a portion of the section 1910a by an overlap distance. In some embodiments, the overlap distance can be at least about 5 mm, at least about 1 cm, at least about 1.5 cm, at least about 2 cm, at least about 2.5 cm, at least about 3 cm, at least about 3.5 cm, at least about 4 cm, or at least about 4.5 cm. In some embodiments, the overlap distance can be no more than about 5 cm, no more than about 4.5 cm, no more than about 4 cm, no more than about 3.5 cm, no more than about 3 cm, no more than about 2.5 cm, no more than about 2 cm, no more than about 1.5 cm, or no more than about 1 cm. Combinations of the above-referenced overlap distances are also possible (e.g., at least about 5 mm and no more than about 5 cm or at least about 2 cm and no more than about 4 cm), inclusive of all values and ranges therebetween. In some embodiments, the overlap distance can be about 5 mm, about 1 cm, about 1.5 cm, about 2 cm, about 2.5 cm, about 3 cm, about 3.5 cm, about 4 cm, about 4.5 cm, or about 5 cm. In embodiments, an adhesive can be disposed on the flap 1914 and/or on the section 1910a in this overlapping region, such that the flag can be secured or coupled to the section 1910a when closed.

As shown, adhesive surfaces 1916 can cover the outer edges of the flap 1914, the outer edges of the section 1910a, and/or the outer edges of the section 1910b. In some embodiments, the adhesive surfaces 1916 can include a reusable or resealable adhesive. A first set of adhesive surfaces 1916 can join or couple the first section 1910a to the second section 1910b. In some embodiments, the adhesive surfaces 1916 can be disposed on the outer edges of the section 1910a and not the section 1910b, and can adhere to a non-adhesive surface on the section 1910b upon coupling the section 1910a to the section 1910b. In some embodiments, the adhesive surfaces 1916 can cover the section 1910b and not the section 1910a, and can adhere to a non-adhesive surface on the section 1910a upon coupling the section 1910a to the section 1910b. In some embodiments, the adhesive surface on the section 1910a can meet the section 1910b at a location above the bottom holder 1980b. In some embodiments, the adhesive surface 1916 on the section 1910a can meet the section 1910b at a location below the bottom holder 1980b. A second set of adhesive surfaces 1916 can join or couple the flap 1914 to the first section 1910a. In some embodiments, the adhesive surface 1916 can cover the outside edges of the flap 1914 and the outer edges of the first section 1910a which overlap with the flap 1914. In some embodiments, the adhesive surface 1916 can cover the outside edges of the flap 1914 and not the outer edges of the first section 1910a, and vice versa.

In some embodiments, the adhesive surfaces 1916 can extend along each outside edge of the flap 1914. As shown, the flap 1914 has a rectangular shape. In some embodiments, the flap 1914 can be circular or oval, or have about 3, about 4, about 5, about 6, about 7, about 9, about 10 or at least about 10 sides, inclusive of all values and ranges therebetween. In some embodiments, the adhesive surfaces 1916 can extend along about 1, about 2, about 3, about 4, or more than about 4 edges of the adhesive surface 1916 (i.e., if the flap 1914 has a non-rectangular shape). As shown, the section 1910a has 6 edges. In some embodiments, the section 1910a can have about 3, about 4, about 5, about 6, about 7, about 8, about 9, about 10 edges, or at least about 10 edges, inclusive of all values and ranges therebetween). As shown, the section 1910b has 6 edges. In some embodiments, the section 1910b can have about 3, about 4, about 5, about 6, about 7, about 8, about 9, about 10, or at least about 10 edges, inclusive of all values and ranges therebetween. In some embodiments, the adhesive surfaces 1916 can extend along about 1, about 2, about 3, about 4, about 5, about 6, or at least about 6 edges of the section 1910a and/or the section 1910b, inclusive of all values and ranges therebetween.

In some embodiments, any of the portions of the adhesive surfaces 1916 can have a width of at least about 1 mm, at least about 2 mm, at least about 3 mm, at least about 4 mm, at least about 5 mm, at least about 6 mm, at least about 7 mm, at least about 8 mm, at least about 9 mm, at least about 1 cm, at least about 2 cm, at least about 3 cm, at least about 4 cm, at least about 5 cm, at least about 6 cm, at least about 7 cm, at least about 8 cm, or at least about 9 cm. In some embodiments, any of the portions of the adhesive surfaces 1916 can have a width of no more than about 10 cm, no more than about 9 cm, no more than about 8 cm, no more than about 7 cm, no more than about 6 cm, no more than about 5 cm, no more than about 4 cm, no more than about 3 cm, no more than about 2 cm, no more than about 1 cm, no more than about 9 mm, no more than about 8 mm, no more than about 7 mm, no more than about 6 mm, no more than about 5 mm, no more than about 4 mm, no more than about 3 mm, or no more than about 2 mm. Combinations of the above-referenced widths are also possible (e.g., at least about 1 mm and no more than about 10 cm or at least about 5 mm and no more than about 2 cm), inclusive of all values and ranges therebetween. In some embodiments, any of the portions of the adhesive surfaces 1916 can have a width of about 1 mm, about 2 mm, about 3 mm, about 4 mm, about 5 mm, about 6 mm, about 7 mm, about 8 mm, about 9 mm, about 1 cm, about 2 cm, about 3 cm, about 4 cm, about 5 cm, about 6 cm, about 7 cm, about 8 cm, about 9 cm, or about 10 cm.

In some embodiments, an adhesive portion 1916 can be disposed at a top or proximal side of the section 1910a, which can be configured to couple or be attached to a patient or dressing, e.g., to hold the sleeve 1910 in place on a patient. In some embodiments, additional adhesive portions 1916 can be disposed on a bottom side of section 1910b (a side of 1910b that faces away from the side 1910a when the two are closed or coupled together), which can be used to secure or attach the sleeve to additional surfaces (e.g., a hospital bed, a table, a ledge, a patient's arm, or other structure).

As shown, a non-adhesive region 1917 of the section 1910a and the section 1910b can be located below one or more adhesive surfaces 1916. While the non-adhesive region 1917 is depicted at the bottom of the sections 1910a, 1910b, it can be appreciated that the non-adhesive region 1917 can be disposed along another side or edge of the sections 1910a, 1910b. The non-adhesive region 1917 allows a user to grasp the first section 1910a and/or second section 1910b and to pull the two apart, e.g., along the perforation. As described above, this can allow for easier access to the medical lines ML and/or quick removal or addition of a medical line ML, without needing to detach the sleeve and/or to insert or feed through a line from under a portion of the sleeve. More specifically, each side of the non-adhesive region 1917 can be grasped and the two sections 1910a, 1910b be pulled apart. In some embodiments, the non-adhesive region 1917 can have a length along the y-axis of at least about 1 mm, at least about 2 mm, at least about 3 mm, at least about 4 mm, at least about 5 mm, at least about 6 mm, at least about 7 mm, at least about 8mm, at least about 9 mm, at least about 1 cm, at least about 2 cm, at least about 3 cm, at least about 4 cm, at least about 5 cm, at least about 6 cm, at least about 7 cm, at least about 8 cm, or at least about 9 cm. In some embodiments, the non-adhesive region 1917 can have a length along the y-axis of no more than about 10 cm, no more than about 9 cm, no more than about 8 cm, no more than about 7 cm, no more than about 6 cm, no more than about 5 cm, no more than about 4 cm, no more than about 3 cm, no more than about 2 cm, no more than about 1 cm, no more than about 9mm, no more than about 8 mm, no more than about 7 mm, no more than about 6 mm, no more than about 5 mm, no more than about 4 mm, no more than about 3 mm, or no more than about 2 mm. Combinations of the above-referenced widths are also possible (e.g., at least about 1 mm and no more than about 10 cm or at least about 5 mm and no more than about 2 cm), inclusive of all values and ranges therebetween. In some embodiments, the non-adhesive region 1917 can have a length along the y-axis of about 1 mm, about 2 mm, about 3 mm, about 4 mm, about 5 mm, about 6 mm, about 7 mm, about 8 mm, about 9 mm, about 1 cm, about 2 cm, about 3 cm, about 4 cm, about 5 cm, about 6 cm, about 7 cm, about 8 cm, about 9 cm, or about 10 cm.

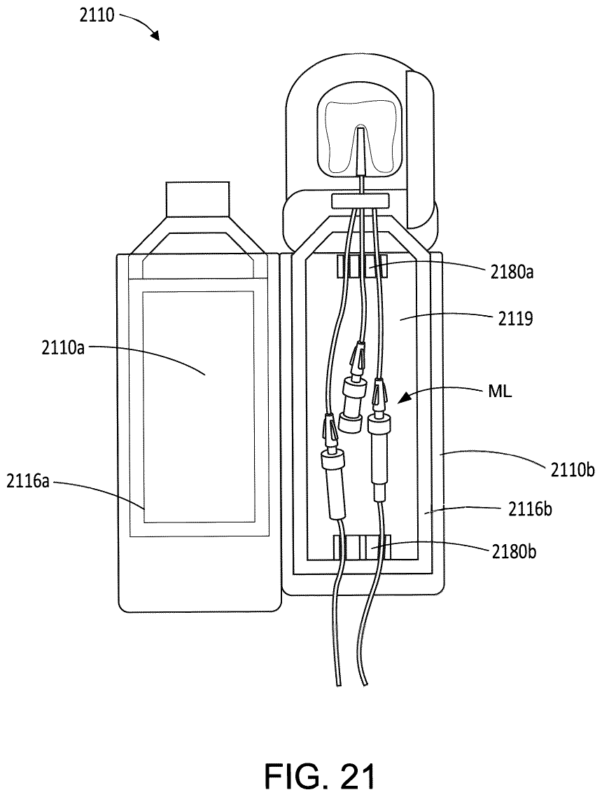

As shown, the holders 1980 are appended or coupled to the section 1910b near the top and the bottom of the section 1910b. In some embodiments, the line holders 1980 can have different sizes and/or have different numbers of slots. In some embodiments, the line holders 1980 can have variable slot sizes where the medical lines ML can be disposed and/or held, depending on what types of medical lines ML are used. For example, a line holder 1980 can have a first slot having a larger width that is configured to receive a thicker or larger line and a second slot having a smaller width that is configured to receive a smaller line. In some embodiments, the material of the holders 1980 can include nonabsorbent foam, silicone, antimicrobial material, or anything similar, e.g., to prevent or help reduce biofilm migration to the medical lines ML. In some embodiments, the holders 1980 can be pre-plugged. In some embodiments, the holders 1980 can be pre-plugged with a plug material that can be removed during the application of the encasement 1910 or placement of lines, allowing the medical care professional to determine if 1, 2, 3, or more medical lines ML are needed. In some embodiments, the holders 1980 can include one or more t-slots, where each of the medical lines ML can be pressed in and retained. For example, a single t-slot can be used to receive multiple lines, in some embodiments, as further described with reference to FIG. 21. While the holders 1980 are shown attached at specific locations to the section 1910b, it can be appreciated that the holders 1980 can be free-floating and placed within unsecured within the sleeve 1910 and/or be couplable (e.g., via adhesive) to the section 1910b during use. For example, the holders 1980 may adhesive backing that can be exposed prior to being attached to the sleeve 1910.

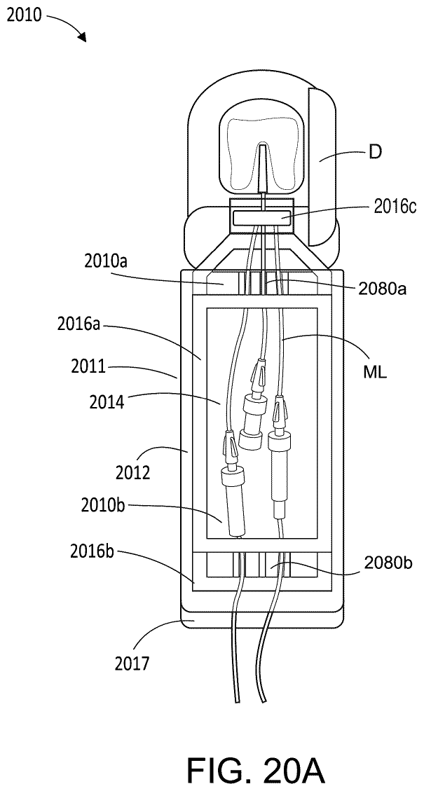

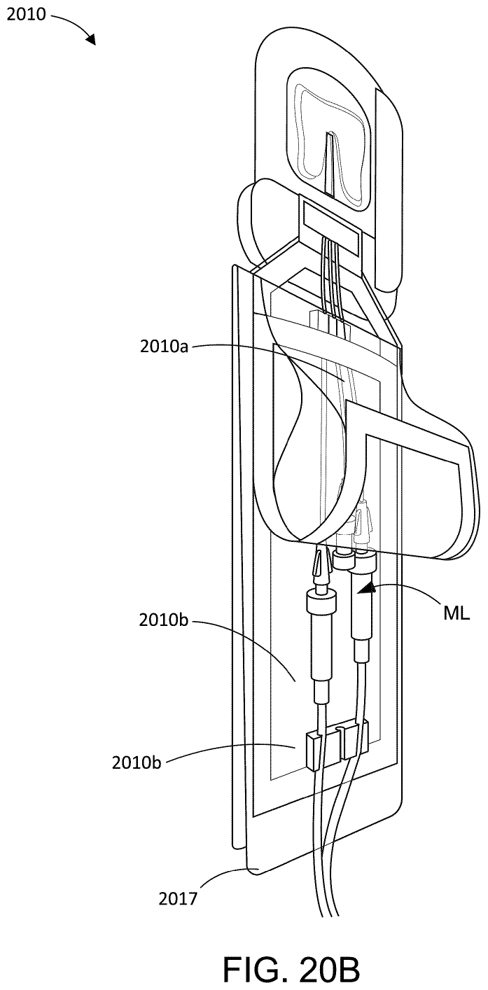

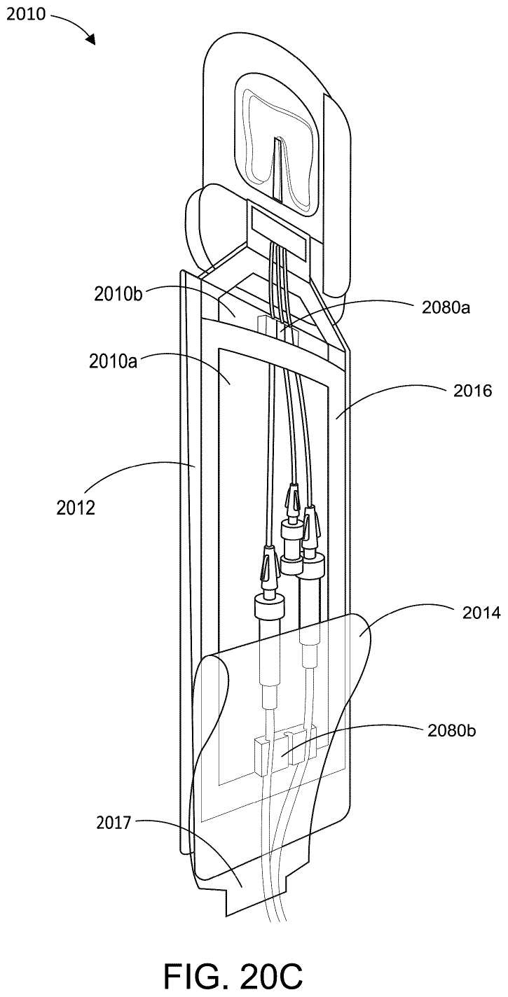

FIGS. 20A-20C are illustrations of an encasement 2010 implemented as a foldable sleeve, according to an embodiment. As shown, the encasement 2010 includes a section 2010a, a section 2010b, a seam 2011, a perforation 2012, a flap 2014, adhesive surfaces 2016a, 2016b (collectively referred to as adhesive surfaces 2016), a non-adhesive region 2017, and holders 2080a, 2080b (collectively referred to as holders 2080). Medical lines ML are included in the encasement 2010. In some embodiments, the section 2010a, the section 2010b, the seam 2011, the perforation 2012, the flap 2014, the adhesive surfaces 2016, the non-adhesive region 2017, and the holders 2080 can be the same or substantially similar to the section 1910a, the section 1910b, the seam 1911, the perforation 1912, the flap 1914, the adhesive surface 1916, the non-adhesive region 1917, and the holders 1980, as described above with respect to FIGS. 19A-19D. Thus, certain aspects of the section 2010a, the section 2010b, the seam 2011, the perforation 2012, the flap 2014, the adhesive surfaces 2016, the non-adhesive region 2017, and the holders 2080 are not described in greater detail herein.

As shown, the various adhesive surfaces 2016a, 2016b, etc. can be disposed at similar locations along the sleeve 2010. In particular, as shown in FIGS. 20A-20C, the adhesive surface 2016c can be configured to attach to a dressing D, e.g., to secure the sleeve in place. As shown in FIG. 20A, the flap 2014 is coupled to the section 2010a. FIG. 20B shows the section 2010a torn along the perforation 2012 and opened. Optionally, in some embodiments, the section 2010a can have an adhesive portion that can be used to hold the section 2010a open. For example, an adhesive portion can be used to secure the section 2010 to another portion of the sleeve 2010 and/or another surface to hold option the section 2010a. FIG. 20C shows the section 2010a coupled to the section 2010b with the flap 2014 opened from the top downward. In some embodiments, the flap 2014 can be opened from the bottom upward.

FIG. 21 is an illustration of an encasement 2110 implemented as a foldable sleeve, according to an embodiment. As shown, the encasement 2110 includes a section 2110a, a section 2110b, adhesive surfaces 2116a, 2116b (collectively referred to as adhesive surfaces 2116), a contrasting surface 2119, and holders 2180a, 2180b (collectively referred to as holders 2180). Medical lines ML are shown in the encasement 2110. In some embodiments, the section 2110a, the section 2110b, the adhesive surfaces 2116, and the holders 2180 can be the same or substantially similar to the section 1910a, the section 1910b, the adhesive surface 1916, and the as described above with reference to FIGS. 19A-19D. Thus, certain aspects of the section 2110a, the section 2110b, the adhesive surfaces 2116, and the holders 2180 are not described in greater detail herein.

As shown, the section 2110b can have a contrasting surface 2119 that is configured to provide a visual contrast to the medical lines ML, e.g., such that the medical lines ML can be easily seen. In some embodiments, the contrasting surface 2119 includes a non-transparent surface applied to the section 2110b.

As shown, the line holder 2080a can include a t-slot, where multiple medical lines have been inserted through a single slot and held between an upper and lower portion of the line holder 2080a. The line holder 2080b can have a different structure from the line holder 2080a. For example, as shown, the line holder 2080b can have multiple slots each for receiving a different medical line.

While certain sections of flexible sleeves described herein are referred to as a “first section,” a “second section,” a “third section,” etc., it can be appreciated that such section designations are intended to identify different sections of the sleeve and are not specific to any one section. For example, a first section of a sleeve can be referred to as a first, second, third, or any numeric designation of a section, and the same applies to a second section, a third section, etc. Moreover, while a second, third, or additional sections may be referred to while describing a particular embodiment, it can be appreciated that the existence of a second, third, or additional section does not necessarily require that there is a certain number of other sections.

Various concepts may be embodied as one or more methods, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

In addition, the disclosure may include other innovations not presently described. Applicant reserves all rights in such innovations, including the right to embodiment such innovations, file additional applications, continuations, continuations-in-part, divisionals, and/or the like thereof. As such, it should be understood that advantages, embodiments, examples, functional, features, logical, operational, organizational, structural, topological, and/or other aspects of the disclosure are not to be considered limitations on the disclosure as defined by the embodiments or limitations on equivalents to the embodiments. Depending on the particular desires and/or characteristics of an individual and/or enterprise user, database configuration and/or relational model, data type, data transmission and/or network framework, syntax structure, and/or the like, various embodiments of the technology disclosed herein may be implemented in a manner that enables a great deal of flexibility and customization as described herein.

All definitions, as defined and used herein, should be understood to control over dictionary definitions, definitions in documents incorporated by reference, and/or ordinary meanings of the defined terms.

As used herein, “proximal” and “distal” refer to locations relative to an entry point of a central line into the patient's body. For example, if point A is located on the central line, 5 cm from the entry point, and point B is located on the central line, 10 cm from the entry point, then point A is proximal to point B and point B is distal to point A.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While specific embodiments of the present disclosure have been outlined above, many alternatives, modifications, and variations will be apparent to those skilled in the art. Accordingly, the embodiments set forth herein are intended to be illustrative, not limiting. Various changes may be made without departing from the spirit and scope of the disclosure. Where methods and steps described above indicate certain events occurring in a certain order, those of ordinary skill in the art having the benefit of this disclosure would recognize that the ordering of certain steps may be modified and such modification are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

1. An apparatus, comprising:
a flexible sleeve configured to receive and enclose a portion of a set of one or more medical lines connected to a patient, the flexible sleeve including:
a first section positionable under the set of medical lines connected to the patient; and
a second section coupled to the first section along a seam, the second section foldable over the first section along the seam and to be reversibly coupled to the first section along a plurality of adhesive sections,
the second section, when reversibly coupled to the first section, configured to enclose the portion of the set of medical lines.

2. The apparatus of claim 1, wherein the first section includes color to provide contrast against the set of medical lines.

3. The apparatus of claim 1, wherein the second section is transparent or translucent to allow the set of medical lines to be visible when the second section is folded over the first section.

4. The apparatus of claim 1, wherein the flexible sleeve further includes a third section that is foldable over the first section.

5. The apparatus of claim 1, wherein the flexible sleeve further includes a window and a flap disposed over the window, the flap configured to reversibly open and close to open and close the window.

6. The apparatus of claim 5, wherein the window is disposed on the second section, and the flap is configured to reversibly couple to the second section.

7. The apparatus of claim 1, wherein at least one of the first section or the second section includes a perforation, the second section configured to be decoupled from the first section by separating the second section from the first section along the perforation.

8. (canceled)

9. (canceled)

10. (canceled)

11. The apparatus of claim 1, further comprising a holder configured to secure one or more medical lines, the holder disposable between the first section and the second section when the second section is reversibly coupled to the first section.

12. (canceled)

13. (canceled)

14. The apparatus of claim 1, wherein the flexible sleeve further includes an adhesive portion configured to couple the flexible sleeve to the patient or a portion of a medical dressing disposed on the patient.

15. The apparatus of claim 1, wherein the flexible sleeve includes an antimicrobial agent coated or embedded thereon.

16. (canceled)

17. An apparatus, comprising:
a flexible sleeve configured to receive and enclose a portion of a set of one or more medical lines connected to a patient, the flexible sleeve including:
a first section positionable under the set of medical lines connected to the patient;
a second section coupled to the first section and foldable over the first section to enclose the portion of the set of lines; and
a flap coupled to the second section, the flap configured to be opened to expose a window that can provide access to the portion of the set of medical lines.

18. (canceled)

19. The apparatus of claim 17, wherein the flap includes an adhesive surface configured to be reversibly coupled to the second section, the adhesive surface configured to seal the window when the flap is closed.

20. (canceled)

21. The apparatus of claim 17, wherein the second section includes a perforation extending along a longitudinal length of the second section.

22. (canceled)

23. (canceled)

24. The apparatus of claim 17, wherein at least one of the first section or the second section further includes an adhesive surface configured to reversibly couple the first section to the second section when the second section is folded over the first section.

25. (canceled)

26. The apparatus of claim 17, wherein the flap includes a transparent section and a non-transparent section.

27. The apparatus of claim 17, further comprising:
a holder configured to secure one or more medical lines, the holder disposable between the first section and the second section when the second section is folded over the first section.

28. (canceled)

29. An apparatus, comprising:
a flexible sleeve configured to receive and enclose a portion of a set of one or more medical lines connected to a patient, the flexible sleeve including:
a first section positionable under the set of medical lines connected to the patient;
a second section coupled to the first section, at least one of the first section or the second section including a first adhesive portion configured to reversibly couple a plurality of edges of the first section to the second section, the second section configured to be folded over the first section such that the first section and second section are reversibly coupled and enclose the portion of the set of medical lines; and
a second adhesive portion disposed on the first section and configured to couple the flexible sleeve to the patient or a medical dressing disposed on the patient.

30. The apparatus of claim 29, wherein the first adhesive portion is disposed along a plurality of edges of the first section.

31. The apparatus of claim 29, wherein the first adhesive portion is disposed a distance away from a distal edge of the at least one of the first section or the second section, such that a distal portion of the first section and the second section can be grasped by a user to decouple the first portion from the second portion.

32. The apparatus of claim 29, wherein at least one of the first section or the second section includes a perforation, the second section configured to be decoupled from the first section by separating the second section from the first section along the perforation.

33. (canceled)

34. (canceled)

35. (canceled)

36. (canceled)
